(19) United States
(12) Patent Application Publication
(10) Pub. No.: US 2023/0295702 A1
(43) Pub. Date: Sep. 21, 2023

(54) METHOD OF DETERMINING LIMIT OF DETECTION AND LIMIT OF QUANTITATION IN NUCLEIC ACID DETECTION TEST

(71) Applicants: Yuuki Yonekawa, Tokyo (JP); Hirotaka Unno, Tokyo (JP); Satoshi Nakazawa, Tokyo (JP); Yunong Ji, Tokyo (JP); Kazusa DNA Research Institute, Kisarazu-shi (JP)

(72) Inventors: Yuuki YONEKAWA, Kanagawa (JP); Hirotaka UNNO, Kanagawa (JP); Satoshi NAKAZAWA, Kanagawa (JP); Yunong JI, Kanagawa (JP); Sakae ITOGA, Kisarazu-shi (JP)

(73) Assignees: Ricoh Company, Ltd., Tokyo (JP); Kazusa DNA Research Institute, Kisarazu-shi (JP)

(21) Appl. No.: 18/147,149

(22) Filed: Dec. 28, 2022

(30) Foreign Application Priority Data
Mar. 18, 2022 (JP) 2022043676

(51) Int. Cl. C12Q 1/6851; C12Q 1/6844

(52) U.S. Cl. CPC C12Q 1/6851; C12Q 1/6844

(57) ABSTRACT

A method of determining a limit of detection and a limit of quantitation in a nucleic acid detection test is provided, the method including, by using a container having a specific configuration, adding a negative specimen that does not contain target nucleic acids to a positive control group and adding a reagent used for a nucleic acid detection test to a negative control group and the positive control group to amplify the target nucleic acids, and determining a smallest specific copy number among specific copy numbers with a detection rate of 95% or greater in the positive control group as a limit of detection and determining a smallest specific copy number among specific copy numbers with CVₗₙ of 35% or less as a limit of quantitation in a case where the target nucleic acids are not detected in the negative control group.

### CROSS-REFERENCE TO RELATED APPLICATIONS

This application claims priority to Japanese Application No. 2022-043676, filed on Mar. 18, 2022, the content of which is hereby incorporated by reference in its entirety.

### BACKGROUND OF THE INVENTION

### Field of the Invention

The present invention relates to a method of determining a limit of detection and a limit of quantitation in a nucleic acid detection test.

### Description of Related Art

Recently, standards for the performance of a polymerase chain reaction (PCR) have been established by the International Organization for Standardization (ISO). ISO15189 and ISO17025 require that whether the validity and performance of equipment used for measurement are maintained be indicated and that maintenance and management be performed. In response to this requirement, for example, Patent Documents 1 and 2 disclose methods of managing the accuracy of an analyzer.

Further, ISO20395 requires setting of limits of detection as performance, and existing genetic testing products are obliged to describe the minimum detection sensitivity.

In the PCR, performance particularly at low copy numbers such as 1000 copies or less cannot be accurately evaluated due to the influence of Poisson distribution. However, in the existing genetic testing products, a copy number of 100 copies or less at which the influence of the Poisson distribution is significant is described as the minimum detection sensitivity. Since the minimum detection sensitivity describes the self-certified value of each institution, the current situation is that the minimum detection sensitivity cannot be objectively and accurately evaluated.

### SUMMARY OF THE INVENTION

The present invention has been made in view of the above-described circumstances and provides a method of determining a limit of detection and a limit of quantitation in a nucleic acid detection test that enables accurate grasping of a limit of detection and a limit of quantitation.

A method of determining a limit of detection and a limit of quantitation in a nucleic acid detection test is provided, the method including, by using a container which includes a negative control group consisting of one or more wells containing no target nucleic acids and a positive control group consisting of wells containing specific copy numbers of target nucleic acids and having three or more wells with different specific copy numbers of the target nucleic acids and in which at least one specific copy number of the target nucleic acids in the positive control group is 1 or greater and less than 100, an amplification step of adding a negative specimen that does not contain the target nucleic acids to the positive control group and adding a reagent used for a nucleic acid detection test to the negative control group and the positive control group to amplify the target nucleic acids, and a determination step of determining a smallest specific copy number among specific copy numbers with a detection rate of 95% or greater in the positive control group as a limit of detection in the nucleic acid detection test and determining a smallest specific copy number among specific copy numbers with CVₗₙ of 35% or less, where CVₗₙ is represented by Equation (I), as a limit of quantitation in the nucleic acid detection test in a case where the target nucleic acids are not detected in the negative control group.

CVₗₙ=\sqrt{(1+E)^{(SD(Cq))}^{²}*^{(ln(1+E)}−1} Equation (I):

(In Equation (I), E represents an efficiency of a nucleic acid amplification reaction. SD (Cq) represents a standard deviation of Cq, and Cq represents the number of cycles at which an amplification curve and a threshold intersect in the nucleic acid amplification reaction.)

According to the determination method of the above-described aspect, it is possible to provide a method of determining a limit of detection and a limit of quantitation in a nucleic acid detection test that enables accurate grasping of a limit of detection and a limit of quantitation.

### BRIEF DESCRIPTION OF THE DRAWINGS

FIG. 1 is a view showing an example of a container used in a determination method according to the present embodiment.

FIG. 2 is a graph showing a relationship between the copy number with variation based on Poisson distribution and the coefficient of variation CV.

FIG. 3 is a schematic view showing an example of an electromagnetic valve type jet head.

FIG. 4 is a schematic view showing an example of a piezo type jet head.

FIG. 5 is a schematic view showing a modified example of the piezo type jet head in FIG. 4.

FIG. 6A is a schematic diagram showing an example of a voltage applied to a piezoelectric element. FIG. 6B is a schematic diagram showing another example of a voltage applied to a piezoelectric element.

FIGS. 7A to 7C are schematic views showing an example of a liquid droplet state.

FIG. 8 is a schematic view showing an example of a dispensing device for sequentially landing liquid droplets in wells.

FIG. 9 is a graph showing an example of a relationship between the frequency of DNA-replicated cells and the fluorescence intensity.

FIG. 10 is a schematic view showing an example of a liquid droplet-forming device.

FIG. 11 is a diagram showing hardware blocks of control means of the liquid droplet-forming device of FIG. 10.

FIG. 12 is a diagram showing functional blocks of the control means of the liquid droplet-forming device of FIG. 10.

FIG. 13 is a flow chart showing an example of an operation of the liquid droplet-forming device.

FIG. 14 is a schematic view showing a modified example of the liquid droplet-forming device.

FIG. 15 is a schematic view showing another modified example of the liquid droplet-forming device.

FIGS. 16A and 16B are views showing a case where a liquid droplet in flight contains two fluorescent particles.

FIG. 17 is a diagram showing a relationship between a brightness value Li in a case where particles do not overlap each other and a brightness value Le that is actually measured.

FIG. 18 is a schematic view showing still another modified example of the liquid droplet-forming device.

FIG. 19 is a schematic view showing even still another example of the liquid droplet-forming device.

FIG. 20 is a schematic view showing an example of a method of counting the number of cells passing through a microchannel.

FIG. 21 is a schematic view showing an example of a method of acquiring an image of a jet head in the vicinity of a nozzle portion.

FIG. 22 is a graph showing a relationship between a probability P (≥2) and an average number of cells.

FIG. 23 is a graph showing a calibration curve for target nucleic acids and endogenous control genes in Experimental Example 1.

FIG. 24 is a graph showing the calibration curve of the target nucleic acids and measurement results of a positive control group measured in the presence of a negative specimen in Experimental Example 1.

FIG. 25 is a graph showing a calibration curve for target nucleic acids and endogenous control genes in Experimental Example 2.

FIG. 26 is a graph showing the calibration curve of the target nucleic acids and measurement results of a positive control group measured in the presence of a negative specimen in Experimental Example 2.

### DETAILED DESCRIPTION OF THE INVENTION

Hereinafter, a method of determining a limit of detection in a nucleic acid detection test according to an embodiment of the present invention (hereinafter, also simply referred to as “determination method of the present embodiment”) will be described with reference to specific embodiments and the accompanying drawings as necessary. Such embodiments and drawings are merely examples for facilitating understanding of the present invention and do not limit the present invention. That is, the shapes, the dimensions, the dispositions, and the like of the members described below can be changed and improved without departing from the scope of the present invention, and the present invention includes equivalents thereof.

Further, in all the drawings, the same constituent elements are denoted by the same reference numerals, and the overlapping description thereof will be omitted as appropriate.

Unless defined otherwise in the present specification, all technical and scientific terms used in the present specification have the same definitions as those commonly understood by those skilled in the art. All patents, applications, other publications, and information referenced in the present specification are hereby incorporated by reference in their entirety. Further, in a case where the publications referenced in the present specification are not consistent with the description in the present specification, the description in the present specification takes precedence.

<<Method of Determining Limit of Detection and Limit of Quantitation in Nucleic Acid Detection Test>>

The determination method of the present embodiment is a method of determining a limit of detection and a limit of quantitation in a nucleic acid detection test by using a container which includes a negative control group consisting of one or more wells containing no target nucleic acids and a positive control group consisting of wells containing specific copy numbers of target nucleic acids and having three or more wells with different specific copy numbers of the target nucleic acids and in which at least one specific copy number of the target nucleic acids is 1 or greater and less than 100.

The determination method of the present embodiment includes the following steps.

The determination method includes an amplification step of adding a negative specimen that does not contain the target nucleic acids to the positive control group and adding a reagent used for a nucleic acid detection test to the negative control group and the positive control group to amplify the target nucleic acids, and a determination step of determining a smallest specific copy number among specific copy numbers with a detection rate of 95% or greater in the positive control group as a limit of detection in the nucleic acid detection test and determining a smallest specific copy number among specific copy numbers with CVₗₙ of 35% or less, where CVₗₙ is represented by Equation (I), as a limit of quantitation in the nucleic acid detection test in a case where the target nucleic acids are not detected in the negative control group.

CVₗₙ=(1+E)^{(SD(Cq))}^{²}*^{ln(1+E)}−1 Equation (I):

(In Equation (1), E represents an efficiency of a nucleic acid amplification reaction, SD (Cq) represents a standard deviation of Cq, and Cq represents the number of cycles at which an amplification curve and a threshold intersect in the nucleic acid amplification reaction.)

In nucleic acid detection tests of the related art, since the limits of detection and the limits of quantitation in the nucleic acid detection tests are not accurately grasped, there is a risk that false-negative specimens are present among the specimens determined to be negative. On the contrary, according to the determination method of the present embodiment, the limit of detection and the limit of quantitation in the nucleic acid detection test can be accurately grasped. Therefore, in a case where a value lower than the limit of detection is detected in the nucleic acid detection test or a value lower than the limit of quantitation is detected, the specimen is not determined to be negative and can be subjected to a nucleic acid detection test that enables more accurate determination with a lower limit of detection or a lower limit of quantitation than that of the nucleic acid detection test, and thus the risk of overlooking false negatives can be reduced. Therefore, the determination method of the present embodiment can also be referred to as a method of suppressing the occurrence of false negatives in specimens provided for nucleic acid detection tests.

Further, the target of the “nucleic acid detection test” in the present specification is a test for detecting target nucleic acids, and the test method is not particularly limited. In a case where the target nucleic acids are specific genes, the nucleic acid detection test can also be referred to as a genetic test. Examples of the nucleic acid detection test include an infectious disease test such as a detection test for SARS-CoV-2 (new coronavirus), a cancer test such as a detection test for epidermal growth factor receptor (EGFR) gene mutation, a food test such as a detection test for pathogen genes such as norovirus or a detection test for recombination genes in food (that is, a detection test for gene-recombination food), and a quality control test for regenerative medicine products such as a detection test for pathogen genes such as mycoplasma.

The term “limit of detection (LOD)” in the present specification denotes a minimum amount (value) that can be detected in a specific nucleic acid detection test. The limit of detection is also referred to as a lower limit of detection.

Further, the term “limit of quantitation (LOQ)” in the present specification denotes a minimum amount (value) that can be quantified in a specific nucleic acid detection test. The limit of quantitation is also referred to as a lower limit of quantitation.

Next, each step in the determination method of the present embodiment will be described in detail below.

<Amplification Step>

In the amplification step, a negative specimen that does not contain the target nucleic acids is added to the positive control group and a reagent used for a nucleic acid detection test is added to the negative control group and the positive control group to amplify the target nucleic acids.

The negative specimen to be added to the positive control group is not limited as long as the specimen does not contain target nucleic acids, and examples thereof include a nucleic acid (DNA or RNA) extract from a heathy subject in an infectious disease test or a cancer test. Alternatively, in a case of a food test, a nucleic acid extract from food that does not contain pathogen genes and a nucleic acid extract from food that is not gene recombination food are exemplary examples. Alternatively, in a case of a quality control test for a regenerative medicine product, nucleic acid extracts from regenerative medicine products that do not contain pathogen genes (for example, stem cells such as ES cells or iPS cells, and cell aggregates thereof) are exemplary examples.

As the method of amplifying target nucleic acids, a known nucleic acid amplification method can be used, and specific examples thereof include a polymerase chain reaction (PCR) method, a UCAN method, and a LAMP method. As the reagent, a reagent typically used in these known amplification methods is appropriately selected and used.

The PCR method is performed using oligonucleotide primers (hereinafter, also simply referred to as “primers”) that hybridize only to the target nucleic acids. Quantitative PCR (qPCR) is preferable as the PCR method. Examples of the quantitative PCR (Q-PCR) include real-time PCR and digital PCR.

The real-time PCR quantifies template nucleic acids based on amplification efficiency by measuring amplification using PCR over time (real time). The quantitation is performed using fluorescent dyes mainly by an intercalation method or a hybridization method.

In the intercalation method, an amplification reaction of template nucleic acids is carried out in the presence of an intercalator that is inserted (intercalated) specifically into double-stranded DNA and emits fluorescence. Examples of the intercalator include SYBR Green I (CAS number: 163795-75-3) and a derivative thereof. Meanwhile, in the hybridization method, a method of using a TaqMan (registered trademark) probe is the most common method, and a probe in which a fluorescent substance and a quenching substance are bound to an oligonucleotide complementary to the target nucleic acid sequence is used.

The digital PCR quantifies the absolute amount of copy numbers by dispersing limiting-diluted (diluted such that the number of target DNA in each microcompartment is 1 or 0) sample DNA into microcompartments to perform PCR amplification and detecting the presence or absence of amplified products in each microcompartment.

The UCAN method is a method that applies the ICAN method, which is an isothermal gene amplification method developed by Takara Bio Inc. The UCAN method uses a DNA-RNA-DNA chimeric oligonucleotide (DRD) as a primer precursor. The DRD primer precursor is designed such that DNA at the 3′ end is modified so that replication of the target nucleic acid due to a DNA polymerase does not occur, and the RNA portion binds to the SNP site. In a case where the DRD primer precursor is incubated with the template, the coexisting RNase H cleaves the RNA portion of the paired DRD primer only in a case where the DRD primer and the target nucleic acid perfectly match each other. In this manner, since the modified DNA is separated from the Y end of the primer to be new, the elongation reaction due to the DNA polymerase proceeds and the template DNA is amplified. Meanwhile, in a case where the DRD primer and the target nucleic acid do not match each other, RNase H does not cleave the DRD primer and amplification of the target nucleic acid does not occur. The amplification reaction after the perfectly matched DRD primer precursor is cleaved by RNase H proceeds by an ICAN reaction mechanism.

The LAMP method defines six regions of the target nucleic acid (F3c, F2c, and F1c from the 3′ end side, and B3. B2, and B1 from the 5′ end side) by the isothermal gene amplification method developed by Eiken Chemical to amplify the regions using four kinds of primers (an FIP primer, an F3 primer, a BIP primer, and a B3 primer) for the six regions.

<Determination Step>

In the determination step, a smallest specific copy number among specific copy numbers with a detection rate of 95% or greater in the positive control group is determined as a limit of detection in the nucleic acid detection test in a case where the target nucleic acids are not detected in the negative control group.

In the determination step, the detection rate can be calculated using the following equation for each specific copy number. In the equation, “UD” denotes “undetermined” (undetected). That is, “number of UDs” denotes the number of wells where target nucleic acids have not been detected.

(detection rate (%))={1−(number of UDs)÷(number of times of measurements)}×100 Equation:

For example, in the positive control group consisting of 1 copy, 2 copies, and 3 copies of RNA, in a case where the detection rate with 1 copy of RNA is 90%, the detection rate with 2 copies of RNA is 100%, and the detection rate with 3 copies of RNA is 1000%, 2 copies of RNA are determined as the limit of detection.

Further, a smallest specific copy number among specific copy numbers with CVₗₙ of 35% or less, which is represented by Equation (I), is determined as a limit of quantitation in the nucleic acid detection test. Further, “CVₗₙ” denotes the coefficient of variation of logarithmic normal distribution data predicted by the concentration repeatedly measured in the amplification reaction of the target nucleic acid.

CVₗₙ=\sqrt{(1+E)^{(SD)Cq))}^{²}*^{ln(1+E)}−1} Equation (I):

(In Equation (I), E represents an efficiency of a nucleic acid amplification reaction, SD (Cq) represents a standard deviation of Cq, and Cq represents the number of cycles at which an amplification curve and a threshold intersect in the nucleic acid amplification reaction.)

For example, in the positive control group consisting of 1 copy, 2 copies, and 3 copies of RNA, in a case where CVₗₙ with 1 copy of RNA is 50%, CVₗₙ with 2 copies of RNA is 33%, and CVₗₙ with 3 copies of RNA is 30%, 2 copies of RNA are determined as the limit of quantitation.

Further, as the method of calculating the limit of detection and the limit of quantitation, the method described in Reference Document 1 (Forootan A et al., “Methods to determine limit of detection and limit of quantification in quantitative real-time PCR (qPCR)”, Biomol Detect Quantif., Vol. 12. pp. 1 to 6, 2017) is referred to.

The container used in the determination method of the present embodiment will be described in detail below.

<Container>

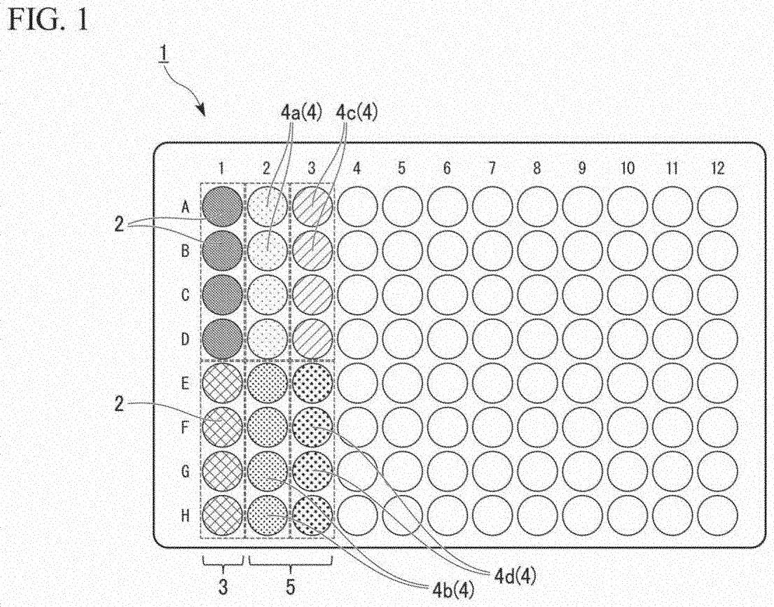

FIG. 1 is a view showing an example of a container used in the determination method according to the present embodiment.

A container 1 includes a negative control group 3 consisting of one or more wells 2 containing no target nucleic acids and a positive control group 5 consisting of wells 4 containing specific copy numbers of target nucleic acids and having three or more wells with different specific copy numbers of the target nucleic acids.

In the negative control group 3, the number of wells containing no target nucleic acids, that is, the number of wells in which the copy number of target nucleic acids is 0 can be set to 1 or greater, 2 or greater, 3 or greater, 4 or greater, 5 or greater, 6 or greater, 7 or greater, 8 or greater, 9 or greater, or 10 or greater. Among these, from the viewpoint of increasing the accuracy in the determination step for the limit of detection, the number of wells in which the copy number of the target nucleic acids is 0 in the negative control group is preferably 4 or greater and more preferably 10 or greater. Further, the upper limit of the number of wells in which the copy number of the target nucleic acids is 0 is not particularly limited and can be set as appropriate according to the number of wells in the container, but can be set to, for example, 20, 15, or 10.

In FIG. 1, a case where the negative control group has four kinds of wells with different specific copy numbers of the target nucleic acid (wells 4a, 4b, 4c, and 4d) is described, and the number of wells with different specific copy numbers of the target nucleic acids is 3 or greater, and can be set to 4 or greater, 5 or greater, 6 or greater, 7 or greater, 8 or greater, 9 or greater, or 10 or greater in the positive control group 5. On the other hand, the upper limit of the number of wells with different specific copy numbers of target nucleic acids is not particularly limited and can be appropriately set according to the number of wells in the container, for example, 20, 15, or 10.

In the positive control group 5, the number of wells with the same specific copy number of the target nucleic acids can be set to, for example, 1 or greater, 2 or greater, 3 or greater, 4 or greater, 5 or greater, 6 or greater, 7 or greater, 8 or greater, 9 or greater, or 10 or greater. Among these, from the viewpoint of increasing the accuracy in the determination step, the number of wells containing the target nucleic acids with the same specific copy number is preferably 4 or greater and more preferably 10 or greater. Meanwhile, in the positive control group 5, the upper limit of wells containing the target nucleic acids with the same specific copy number is not particularly limited, and can be set as appropriate according to the number of wells in the container, for example, 20, 15, or 10.

At least one, preferably at least three, and more preferably at least five specific copy numbers of the target nucleic acids are 1 or greater and less than 100, and the copy number can be appropriately set within a range of 1 or greater and less than 100, for example, 1, 2, 3, 4, 5, 6, 7, 8, 9, 10, 15, 20, 25, 30, 40, 50, 60, 70, 80, or 90.

Although not shown in FIG. 1, the container 1 further includes a well group for creating a calibration curve, which consists of wells containing specific copy numbers of target nucleic acids and has three or more wells with different specific copy numbers of the target nucleic acids. Further, the well group for creating a calibration curve does not contain the negative specimen. In this manner, the inhibitory effect of the negative specimen on the target nucleic acids in the positive control group can be evaluated by creating an accurate calibration curve for the target nucleic acids, and thus the lower limit of quantitation can be more accurately determined.

In the well group for creating a calibration curve, the number of wells with different specific copy numbers of the target nucleic acids can be set to 3 or greater, 4 or greater, 5 or greater, 6 or greater, 7 or greater, 8 or greater, 9 or greater, or 10 or greater. Meanwhile, the upper limit of the number of wells with different specific copy numbers of nucleic acids for creating a calibration curve is not particularly limited, and can be appropriately set according to the number of wells of a device, for example, 20, 15, or 10.

In the well group for creating a calibration curve, at least one specific copy number of the target nucleic acids is 1 or greater and less than 100, and the copy number can be appropriately set within a range of 1 or greater and less than 100, for example, 1, 2, 3, 4, 5, 6, 7, 8, 9, 10, 15, 20, 25, 30, 40, 50, 60, 70, 80, or 90.

The specific copy number of the target nucleic acids in the positive control group and the specific copy number of the target nucleic acids in the well group for creating a calibration curve may be the same as or different from each other, but are preferably different from each other.

Further, in a case where the specific copy number of the target nucleic acids in the positive control group and the specific copy number of the target nucleic acids in the well group for creating a calibration curve are different from each other, and the specific copy number of one target nucleic acid in the positive control group is set as X₁ and the specific copy numbers of two target nucleic acids in the well group for creating a calibration curve, which are copy numbers different from each other and adjacent to each other, are set as Y₁ and Y₂, it is more preferable that X₁, Y₁, and Y₂ have a relationship represented by Expression of “Y₁<X₁<Y₂”.

In a case where the specific copy number of the target nucleic acids in the positive control group and the specific copy number of the target nucleic acids in the well group for creating a calibration curve have the above-described relationship, the inhibitory effect of the negative specimen on the target nucleic acids in the positive control group can be more accurately evaluated, and the lower limit of quantitation can be more accurately determined.

In the well group for creating a calibration curve, the number of wells containing the target nucleic acids with the same specific copy number can be set to 2 or greater, 3 or greater, 4 or greater, 5 or greater, 6 or greater, 7 or greater, 8 or greater, 9 or greater, or 10 or greater. Among these, from the viewpoint of increasing the accuracy of the uncertainty of the quantitation value of the target nucleic acids, it is preferable that the well group include 3 or greater wells containing the target nucleic acid with the same specific copy number.

Meanwhile, in the well group for creating a calibration curve, the upper limit of the number of wells containing the target nucleic acids with the same specific copy number is not particularly limited, and can be appropriately set according to the number of wells of the device, for example, 20, 15, or 10.

[Specific Copy Number]

In the present specification, the expression “copy number of the nucleic acids present in the well is specified” denotes that the number of nucleic acids present in the well is specified with a certain degree of accuracy or higher. That is, it can be said that the number of nucleic acids actually present in the well is known.

In other words, the specific copy number in the present specification is a value that has higher accuracy and higher reliability as a number than a predetermined copy number (calculated estimated value) obtained by serial dilution of the related art and that is controlled regardless of Poisson distribution particularly even in a region with a low copy number of 1,000 or less.

It is preferable that the controlled value be set such that the coefficient of variation CV, which represents the uncertainty, is generally within the range of any of CV <1/√x or CV≤20% with respect to the average copy number x.

Here, “copy number” and “number of molecules” of nucleic acids are associated with each other in some cases. Specifically, for example, in a case of a G1 phase yeast in which a base sequence of a nucleic acid (single-stranded) has been introduced into two sites on the genome, the number of molecules of the nucleic acid (the number of identical chromosomes) is 1 and the copy number of the nucleic acid is 2 in a case where the number of yeasts is 1. In the present specification, the specific copy number of the nucleic acids is also referred to as the absolute number of the nucleic acids.

In the determination method of the present embodiment, in a case where a plurality of reaction spaces containing target nucleic acids (hereinafter, also referred to as “wells”) are present, the expression “copy number of each nucleic acid contained in each well is identical” denotes that the variation in the number of nucleic acids that occurs in a case of filling the reaction spaces with each nucleic acid is within an acceptable range. Whether or not the variation in the number of nucleic acids is within an acceptable range can be determined based on the information of uncertainty shown below.

Examples of the information related to the specific copy numbers of the nucleic acids include information of uncertainty and information of nucleic acids.

The term “uncertainty” is defined as “parameter, associated with the result of a measurement, that characterizes the dispersion of the values that could reasonably be attributed to the measurand” in ISO/IEC Guide 99:2007 [International vocabulary of metrology-basic and general concepts and associated terms (VIM)].

Here, “values that could reasonably be attributed to the measurand” denotes candidates for the true values of the measurand. In other words, the uncertainty denotes information related to variations in measurement results due to operations, equipment, and the like involved in production of the object to be measured. As the uncertainty increases, the variation to be expected as a measurement result increases. The uncertainty may be, for example, the standard deviation obtained from the measurement result or may be a half value of the confidence level expressed as the width of the value in which the true value is included with a predetermined probability or more.

The uncertainty can be calculated based on the Guide to the Expression of Uncertainty in Measurement (GUM: ISO/IEC Guide 98-3) and the guidelines on uncertainty of measurement in Japan Accreditation Board Note 10 tests.

As the method of calculating the uncertainty, for example, two methods, a type A evaluation method of using statistics such as measured values and a type B evaluation method of using information related to uncertainty obtained from calibration certificates, manufacturer's specifications, published information, and the like can be applied.

The uncertainty can be expressed with the same confidence level by converting all uncertainty obtained from factors such as operations and measurements into standard uncertainty. The standard uncertainty denotes the variation in the average value obtained from the measured values.

As an example of the method of calculating uncertainty, for example, factors that cause uncertainty are extracted and the uncertainty (standard deviation) of each factor is calculated. Subsequently, the calculated uncertainty of each factor is combined by the sum of squares method to calculate the combined standard uncertainty. Since the sum of squares method is used in the calculation of the combined standard uncertainty, factors with sufficiently small uncertainty among the factors that cause uncertainty can be ignored.

As the information related to uncertainty, the coefficient of variation of the nucleic acid-filling wells may be used. The coefficient of variation denotes, for example, the relative value of variation in the number of nucleic acids filling each well that occurs in a case where the wells are filled with nucleic acids. That is, the coefficient of variation denotes the filling accuracy of the number of nucleic acids filling each well. The coefficient of variation is a value obtained by dividing a standard deviation a by an average value x. Here, in a case where the value obtained by dividing the standard deviation a by an average copy number (average filling copy number) x is defined as a coefficient of variation CV, the relational expression of Equation 1 is obtained.

CV=σ/x (Equation 1)

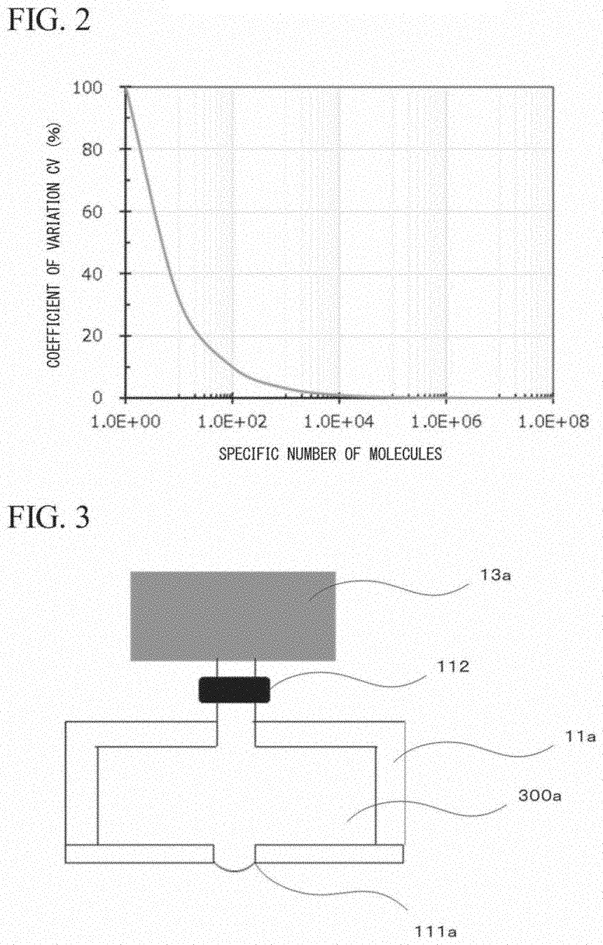

In general, nucleic acids are in a state of being randomly distributed in a sample in Poisson distribution. Therefore, in the serial dilution method, that is, in the randomly distributed state in the Poisson distribution, the standard deviation a can be considered to satisfy the average copy number x and the relational expression of Equation 2. In this manner, in a case where a sample containing nucleic acids is diluted by the serial dilution method, and the coefficient of variation CV (CV value) of the average copy number x is acquired from the standard deviation a and the average copy number x using Equation 3 derived from Equation 1 and Equation 2, the results are as shown in Table 1 and FIG. 2. The CV value of the coefficient of variation of the copy number with variation based on the Poisson distribution can be acquired from FIG. 2.

$$σ = \sqrt{x} \qquad ( Equation 2 )$$

$$C V = \frac{1}{\sqrt{x}} \qquad ( Equation 3 )$$

TABLE 1

| Average copy number x | Coefficient of variation CV |
|---|---|
| 1.00E+00 | 100.00% |
| 1.00E+01 | 31.62% |
| 1.00E+02 | 10.00% |
| 1.00E+03 | 3.16% |
| 1.00E+04 | 1.00% |
| 1.00E+05 | 0.32% |
| 1.00E+06 | 0.10% |
| 1.00E+07 | 0.03% |
| 1.00E+08 | 0.01% |

As shown in the results of Table 1 and FIG. 2, for example, in a case where wells are filled with 100 copies of nucleic acids by the serial dilution method, the copy number of the nucleic acids finally filling the wells has at least 10% of the coefficient of variation (CV value) even in a case where other accuracies are ignored.

In the copy number of the nucleic acids, the CV value of the coefficient of variation and the average specific copy number x of the nucleic acids preferably satisfy “CV<1/√x” and more preferably “CV<½√x”.

As the information related to uncertainty, in a case where a plurality of wells containing nucleic acids are present, it is preferable to use the information related to uncertainty for all the wells based on the specific copy numbers of the nucleic acids contained in the wells.

There are several factors that cause uncertainty, and examples of such factors include, in a case where nucleic acids are introduced into cells and the cells are counted and dispensed into wells, the number of nucleic acids in the cells (for example, the cell cycle of cells), means for disposing the cells in wells (including the results of the operation of each part of an ink jet device or a device that controls the timing of the operation of the ink jet device, for example, the number of cells contained in liquid droplets in a case where a cell suspension is formed into liquid droplets), the frequency at which cells are disposed in appropriate positions in the wells (for example, the number of cells disposed in the wells), and contamination (mixing of contaminants, hereinafter, also referred to as “contamination”) due to mixing of nucleic acids into the cell suspension caused by destruction of cells in the cell suspension.

Examples of information related to nucleic acids include information related to the specific copy number of the nucleus. Examples of information related to the specific copy numbers of the nucleic acids include information related to uncertainty of the specific copy numbers of the nucleic acids contained in the wells.

In the determination method of the present embodiment, it is preferable that the variation in the specific copy number of the target nucleic acids be less than or equal to the variation in the specific copy number of the target nucleic acids. In this manner, the limit of detection in the nucleic acid detection test can be grasped with higher accuracy.

[Target Nucleic Acid]

The nucleic acid generally denotes an organic compound of a polymer to which a nitrogen-containing base derived from purine or pyrimidine, sugar, and phosphoric acid are regularly bound, and also includes a nucleic acid analog. The nucleic acid is not particularly limited and can be appropriately selected depending on the purpose, and examples thereof include DNA, RNA, and cDNA.

The target nucleic acid may be a nucleic acid fragment or may be incorporated into a nucleus of a cell, and it is preferable that the target nucleic acid be incorporated into a nucleus of a cell. Therefore, the target nucleic acid is used in a state of being extracted from a cell. That is, it is preferable that each target nucleic acid be a nucleic acid extracted from a cell.

The target nucleic acid may be a natural product obtained from an organism, a processed product thereof, a product produced by using a gene-recombination technology, or an artificially synthesized nucleic acid that has been chemically synthesized. These may be used alone or in combination of two or more kinds thereof.

The target nucleic acid sequence may be a sequence derived from any of a eukaryote, a prokaryote, a multicellular organism, or a unicellular organism. Examples of the eukaryote include animals, insects, plants, fungi, algae, and protozoa. As the animals, for example, vertebrates such as fish, amphibians, reptiles, birds, and mammals are preferable.

Among vertebrates, mammals are more preferable. Examples of mammals include humans, monkeys, marmosets, dogs, cows, horses, sheep, pigs, rabbits, mice, rats, guinea pigs, and hamsters. Among these, humans are preferable.

Among the examples of the target nucleic acid, it is preferable that the target nucleic acid be human genomic DNA or a fragment thereof.

The artificially synthesized nucleic acid denotes a nucleic acid obtained by artificially synthesizing a nucleic acid consisting of constituent components (a base, deoxyribose, and phosphoric acid) identical to those of DNA or RNA that are naturally present. The artificially synthesized nucleic acid may be, for example, a nucleic acid having a base sequence that encodes a protein, or a nucleic acid having any base sequence.

Examples of the analog of a nucleic acid or a nucleic acid fragment include a nucleic acid or nucleic acid fragment bound to a non-nucleic acid component, a nucleic acid or nucleic acid fragment labeled with a labeling agent such as a fluorescent dye or an isotope (such as a primer or probe labeled with a fluorescent dye or a radioactive isotope), and an artificial nucleic acid obtained by changing the chemical structure of some nucleotides constituting a nucleic acid or nucleic acid fragment (such as PNA. BNA, or LNA).

The form of the target nucleic acid is not particularly limited and can be appropriately selected depending on the purpose thereof, and examples thereof include nucleic acids such as a double-stranded nucleic acid, a single-stranded nucleic acid, and a partially double-stranded or single-stranded nucleic acid, and the target nucleic acid may be a circular or linear plasmid. Further, the target nucleic acid may have modifications or mutations.

It is preferable that the target nucleic acid have a specific base sequence with a known base sequence. The specific base sequence is not particularly limited and can be appropriately selected depending on the purpose thereof, and examples thereof include a base sequence used for a genetic disease test, a non-natural base sequence that does not exist in nature, a base sequence derived from animal cells, a base sequence derived from plant cells, a base sequence derived from fungal cells, a base sequence derived from bacteria, and a base sequence derived from viruses. These may be used alone or in combination of two or more kinds thereof.

The target nucleic acid may be a nucleic acid derived from cells used or a nucleic acid introduced by gene introduction. The number of kinds of target nucleic acids may be one or more. In a case where a nucleic acid that has been incorporated into the nucleus of a cell by gene introduction is used as the target nucleic acid, it is preferable to confirm that the specific copy number (for example, 1 copy) of the target nucleic acid has been introduced into one cell. The method of confirming that the specific copy number of the target nucleic acid has been introduced is not particularly limited, and can be appropriately selected depending on the purpose thereof, and examples thereof include a sequencing method, a PCR method, and a Southern blotting method.

In a case where the target nucleic acid is introduced into the nucleus of a cell, the method of gene introduction is not particularly limited as long as the desired copy number of the specific nucleic acid sequence can be introduced to a desired location, and examples thereof include homologous recombination, CRISPR/Cas9, CRISPR/Cpf1, TALEN, Zinc finger nuclease, Flip-in, and Jump-in. Alternatively, the target nucleic acid may be introduced into the nucleus of a cell in the form of a plasmid, an artificial chromosome, or the like. For example, in a case where yeasts (yeast cells) are used as cells, homologous recombination is preferable among the examples from the viewpoints of high efficiency and ease of control.

The target nucleic acid may be microcompartmentalized in the sample by microdomains or carriers. Here, the target nucleic acid microcompartmentalized by the microdomains or carriers may be one copy or more copies. Further, in a case where two or more copies of the target nucleic acids are microcompartmentalized, a plurality of the target nucleic acids may consist of the same sequence or different sequences. In a case where the target nucleic acid is microcompartmentalized by a carrier in the sample, the target nucleic acid is present in a state of being bound to the carrier directly or indirectly via a linker or the like.

Examples of the form of the microdomains include cells, liposomes, microcapsules, viruses, droplets, and emulsions. Examples of the form of the carriers include metal particles, magnetic particles, ceramic particles, polymer particles, and protein particles.

[Cell]

A cell is a structural and functional unit that forms an organism. The cell containing the target nucleic acid is not particularly limited and can be appropriately selected depending on the purpose thereof, and examples thereof include eukaryotic cells, prokaryotic cells, multicellular organism cells, and unicellular organism cells. The cell may be used alone or in combination of two or more kinds thereof.

The eukaryotic cells are not particularly limited and can be appropriately selected depending on the purpose thereof, and examples thereof include animal cells, insect cells, plant cells, fungal cells, algae, and protozoa. These may be used alone or in combination of two or more kinds thereof. Among these, animal cells or fungal cells are preferable.

Examples of animals from which animal cells are derived include fish, amphibians, reptiles, birds, and mammals. Among these, mammals are preferable. Examples of mammals include humans, monkeys, marmosets, dogs, cows, horses, sheep, pigs, rabbits, mice, rats, guinea pigs, and hamsters. Among these, humans are preferable.

Animal cells may be adherent cells or suspended cells. The adherent cells may be primary cells directly collected from a tissue or an organ, primary cells directly collected from a tissue or an organ that have been subcultured for several generations, or differentiated cells or undifferentiated cells.

The differentiated cells are not particularly limited and can be appropriately selected depending on the purpose thereof, and examples thereof include endothelial cells such as hepatocytes, which are the parenchymal cells of the liver, stellate cells, Kupffer cells, vascular endothelial cells, sinusoidal endothelial cells, and corneal endothelial cells: epidermal cells such as fibroblasts, osteoblasts, osteoclasts, periodontal ligament-derived cells, and epidermal keratinocytes; epithelial cells such as tracheal epithelial cells, gastrointestinal epithelial cells, cervical epithelial cells, and corneal epithelial cells; mammary gland cells, pericytes; muscle cells such as smooth muscle cells and myocardial cells, renal cells, and pancreatic islet of Langerhans cells; nerve cells such as peripheral nerve cells and optic nerve cells; chondrocytes, and bone cells.

The undifferentiated cells are not particularly limited and can be appropriately selected depending on the purpose thereof, and examples thereof include totipotent stem cells such as embryonic stem cells (ES cells) and induced pluripotent stem cells (iPS cells): pluripotent cells such as mesenchymal stem cells; and unipotent stem cells such as endothelial progenitor cells.

The fungal cells are not particularly limited and can be appropriately selected depending on the purpose thereof, and examples thereof include mold and yeasts. These may be used alone or in combination of two or more kinds thereof. Among these, yeasts are preferable from the viewpoint that the cell cycle can be regulated and a haploid can be used. The cell cycle denotes a process in which cell division occurs in a case where cells multiply, and cells (daughter cells) produced by the cell division become cells (mother cells) that undergo cell division again to produce new daughter cells.

The yeasts are not particularly limited and can be appropriately selected depending on the purpose thereof, and preferred examples thereof include yeasts which are synchronously cultured in synchronism with the G0/G1 phase and fixed in the G1 phase. Further, as the yeasts, for example, Bar1 gene-deficient yeasts with increased sensitivity to pheromones (sex hormones) that control the cell cycle in the G1 phase are preferable. In a case where the yeasts are Bar1 gene-deficient yeasts, the abundance ratio of yeasts whose cell cycle cannot be controlled can be reduced, and thus an increase in the copy number of nucleic acids in cells can be prevented.

The prokaryotic cells are not particularly limited and can be appropriately selected depending on the purpose thereof, and examples thereof include eubacteria such as Escherichia coli and archaebacteria. These may be used alone or in combination of two or more kinds thereof.

It is preferable that the cells be dead cells. In a case where the cells are dead cells, it is possible to prevent the amount of intracellular nucleic acids from changing due to the occurrence of cell division after fractionation. It is preferable that the cells be capable of emitting light when receiving light. In a case of the cells being capable of emitting light when receiving light, the number of cells can be controlled with high accuracy so that the cells can be landed in the wells.

It is preferable that the cells be capable of emitting light when receiving light. Receiving light denotes that the cells receive light. The light emission of cells is detected by an optical sensor. The optical sensor denotes a passive sensor that collects any of visible light that can be seen by the human eye, near-infrared rays with longer wavelengths, short-wave infrared rays, and light up to the thermal infrared region by a lens and acquires the shape and the like of target cells as image data.

The cells that can emit light when receiving light are not particularly limited and can be appropriately selected depending on the purpose thereof, and examples thereof include cells stained by a fluorescent dye, cells expressing fluorescent protein, and cells labeled with a fluorescence-labeled antibody. The site stained with a fluorescent dye, the site expressing fluorescent protein, and the site labeled with a fluorescence-labeled antibody in cells are not particularly limited, and examples thereof include whole cells, cell nuclei, and cell membranes.

Examples of the fluorescent dye include fluoresceins, azos, rhodamines, coumarins, pyrenes, and cyanines. These may be used alone or in combination of two or more kinds thereof. Among these, fluoresceins, azos, rhodamines, or cyanines are preferable, and Eosin, Evans Blue, Trypan Blue, Rhodamine 6G, Rhodamine B, Rhodamine 123, or Cy3 is more preferable.

Commercially available products can be used as fluorescent dyes, and examples of the commercially available products include EosinY (trade name, manufactured by FUJIFILM Wako Pure Chemical Corporation), Evans Blue (trade name, manufactured by FUJIFILM Wako Pure Chemical Corporation), Trypan Blue (trade name, manufactured by FUJIFILM Wako Pure Chemical Corporation), Rhodamine 6G (trade name, manufactured by FUJIFILM Wako Pure Chemical Corporation), Rhodamine B (trade name, manufactured by FUJIFILM Wako Pure Chemical Corporation), and Rhodamine 123 (trade name, manufactured by FUJIFILM Wako Pure Chemical Corporation).

Examples of the fluorescent protein include Sirius, EBFP, ECFP, mTurquoise, TagCFP, AmCvan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, Red, KillerRed, mCherry, mPlum, PS-CFP, Dendra2, Kaede, EosFP, and KikumeGR. These may be used alone or in combination of two or more kinds thereof.

The fluorescence-labeled antibody is not particularly limited as long as the antibody can bind to a target cell and is fluorescently labeled, and can be appropriately selected depending on the purpose thereof, and examples thereof include an FITC-labeled anti-CD4 antibody and a PE-labeled anti-CD8 antibody. These may be used alone or in combination of two or more kinds thereof.

The volume-average particle diameter of the cells in a free state is preferably 30 μm or less, more preferably 10 μm or less, and particularly preferably 7 μm or less. In a case where the volume-average particle diameter of the cells is 30 μm or less, the cells can be suitably used for the liquid droplet-jetting means such as an ink jet method or a cell sorter.

The volume-average particle diameter of cells can be measured, for example, by the following measuring method. In a case where yeasts are used as cells, the volume-average particle diameter thereof can be measured by taking out 10 μL of the liquid from the prepared stained yeast dispersion liquid, placing the liquid on a PMMA plastic slide, and performing measurement using an automatic cell counter (trade name. Countess Automated Cell Counter, manufactured by Invitrogen Corp.). Further, the number of cells can also be acquired by the same measuring method as described above.

The density of cells in the cell suspension is not particularly limited, and can be appropriately selected depending on the purpose, but is preferably 5×10⁴ cells/mL or greater and 5×10⁸ cells/mL or less and more preferably 5×10⁴ cells/mL or greater and 5×10⁷ cells/mL or less. In a case where the cell density is in the above-described ranges, the jetted liquid droplets can reliably contain the cells. The cell density can be measured using an automatic cell counter (trade name: Countess Automated Cell Counter, manufactured by Invitrogen Corp.) or the like in the same manner as in the method of measuring the volume-average particle diameter.

The copy numbers of the target nucleic acids contained in the cells can be set to, for example, 1, 2, 3, 4, 5, 6, 7, 8, 9, 10, 15, 20, 30, 50, 100 copies. Further, in a case where two or more of a plurality of cells are added to wells, the copy numbers of the nucleic acids contained in the cells may be the same as or different from each other, but it is preferable that the copy numbers thereof be the same as each other from the viewpoint that the container used in the determination method of the present embodiment can be efficiently produced.

Among these, it is preferable that one cell contain one copy or one molecule of the target nucleic acid. In this case, “number of cells” can be defined as “total copy number or total number of molecules of target nucleic acids present in the wells”, and thus the total copy number or the total number of molecules of the target nucleic acids present in the wells can be more simply grasped.

Further, in the determination method of the present embodiment, as the cells containing the target nucleic acids, forms other than the cells can be used in place of the cells described above as long as the same conditions as the conditions for the cells can be reproduced. Examples of the forms other than cells include liposomes, microcapsules, viruses, droplets, and emulsions.

(Liposome)

A liposome is a lipid vesicle formed of a lipid bilayer containing lipid molecules and specifically denotes a vesicle containing closed lipids having a space separated from the outside by the lipid bilayer that occurs based on the polarity of a hydrophobic group and a hydrophilic group of the lipid molecules.

The liposome is a closed vesicle formed of a lipid double membrane for which lipids are used, and has a water phase (internal water phase) within the space of the closed vesicle. The internal water phase contains water and the like. The liposome may be single lamellar (single layer lamellar or unilamellar, which is a single bilayer membrane) or multilamellar (multilamellar, multiple bilayer membranes with an onion-like structure, and each layer is partitioned with a water-like layer).

The form of the liposome is not particularly limited as long as the liposome can include a nucleic acid. The term “include” denotes that the nucleic acid has a form of being contained in the internal water phase or in the membrane with respect to the liposome. Examples of the form include a form in which a nucleic acid is enclosed in a closed space formed of a membrane, a form in which a nucleic acid is included in the membrane itself, and a combination thereof.

The size (average particle diameter) of the liposomes is not particularly limited as long as the size enables inclusion of the nucleic acid. Further, a spherical shape or a shape close to a spherical shape is preferable as the shape of the liposome.

The component (membrane component) constituting the lipid bilayer of the liposome is selected from the lipid. Any lipid can be used as long as the lipid is dissolved in a mixed solvent of a water-soluble organic solvent and an ester-based organic solvent. Specific examples of the lipid include phospholipids, lipids other than the phospholipids, cholesterols, and derivatives thereof. The component may be used alone or in combination of two or more kinds thereof.

(Microcapsule)

A microcapsule denotes a minute particle having a wall material and a hollow structure and can include a nucleic acid in the hollow structure. The microcapsule is not particularly limited, and the wall material, the size, and the like can be appropriately selected depending on the purpose thereof.

Examples of the wall material of the microcapsule include a polyurethane resin, polyurea, a polyurea-polyurethane resin, a urea-formaldehyde resin, a melamine-formaldehyde resin, polyamide, polyester, polysulfonamide, polycarbonate, polysulfinate, epoxy, acrylic acid ester, methacrylic acid ester, vinyl acetate, and gelatin. These may be used alone or in combination of two or more kinds thereof.

The size of the microcapsule is not particularly limited as long as the size enables inclusion of the nucleic acid, and can be appropriately selected depending on the purpose thereof. The method of producing the microcapsule is not particularly limited and can be appropriately selected depending on the purpose thereof, and examples thereof include an in-situ method, an interfacial polymerization method, and a coacervation method.

[Other Components]

Each well of the negative control group may contain a reagent used in the treatment and the pretreatment in the nucleic acid detection test.

Similarly, even each well of the positive control group may contain a reagent used in the treatment and the pretreatment of the nucleic acid detection test in addition to the target nucleic acid.

Examples of the reagent include primers and amplification reagents. In a case of the polymerase chain reaction (PCR), the primer is a synthetic oligonucleotide having a complementary base sequence of 18 to 30 bases specific to the template DNA (target nucleic acid in the present embodiment), and two sites (one pair) of a forward primer (sense primer) and a reverse primer (antisense primer) are set to sandwich an amplification target region.

Examples of the amplification reagent in the case of the polymerase chain reaction (PCR) include DNA polymerase as an enzyme, four kinds of bases (dGTP, dCTP, dATP, and dTTP) as substrates, Mg²⁺ (magnesium chloride with a final concentration of approximately 1 mM or greater and 2 mM or less), and a buffer that maintains the optimum pH (pH of 7.5 to 9.5).

The state of the target nucleic acid in the reaction space and the state of the primer and the amplification reagent in a case of being present are not particularly limited, and can be appropriately selected depending on the purpose thereof. For example, the target nucleic acid, the primer, and the amplification reagent may be in any of a solution or solid state.

From the viewpoint of usability, the solution state is particularly preferable. In a case where the target nucleic acid, the primer, and the amplification reagent are in a solution state, these can be immediately used for the test by a user. From the viewpoint of transportation, the solid state is particularly preferable, and a solid dry state is more preferable. In a case where the target nucleic acid, the primer, and the amplification reagent are in a solid dry state, the decomposition rate of the reagent by a degrading enzyme or the like can be reduced, and the preservability of the reagent can be improved.

The drying method is not particularly limited and can be appropriately selected depending on the purpose thereof, and examples thereof include freeze drying, heat drying, hot air drying, vacuum drying, steam drying, suction drying, infrared drying, barrel drying, and spin drying.

It is preferable that the reaction space contain an appropriate amount of reagent such that the reagent in a solid dry state can be immediately used as a reaction solution by being dissolved in a buffer or water immediately before the use of the container.

The container may contain the target nucleic acids or the primers and the amplification reagents described above in all of the plurality of reaction spaces, or may contain the target nucleic acids or the primers and the amplification reagents described above in some of the plurality of reaction spaces. In the latter case, the remaining reaction spaces may, for example, be empty or contain reagents with different compositions.

In the container, the form of the reaction space is not particularly limited, and examples thereof include wells, liquid droplets, and compartments on a substrate. For example, in a case where the reaction space is a well, the container may be in the form of a well plate.

[Well]

The shape, the number, the volume, the material, and the color of the wells are not particularly limited and can be appropriately selected depending on the purpose thereof. The shape of the well is not particularly limited as long as the well can contain the target nucleic acid and, in a case of being present, the reagent and can be appropriately selected depending on the purpose thereof, and examples thereof include recessed portions with a flat bottom, a round bottom, a U-shaped bottom, a V-shaped bottom, and the like.

A plurality of wells are present, and the number thereof is preferably 5 or more and more preferably 50 or greater. A multi-well plate having two or more wells is suitably used. Examples of the multi-well plate include well plates having, for example, 24, 48, 96, 384, or 1,536 wells.

The volume of the well is not particularly limited and can be appropriately selected depending on the purpose thereof, but is preferably 10 μL or greater and 1,000 μL or less in consideration of the sample volume used in typical quantitative PCR.

The material of the well is not particularly limited and can be appropriately selected depending on the purpose thereof, and examples thereof include polystyrene, polypropylene, polyethylene, a fluororesin, an acrylic resin, polycarbonate, polyurethane, polyvinyl chloride, and polyethylene terephthalate.

The color of the well may be, for example, transparent, translucent, colored, completely opaque, or the like. The wettability of the well is not particularly limited and can be appropriately selected depending on the purpose thereof, and for example, the well may be water repellent. In a case where the wettability of the well is water repellent, adsorption of the reagent on the inner wall of the well can be reduced. Further, in the case where the wettability of the well is water repellent, the reagent in the well can easily move in a solution state.

The method of making the inner wall of the well water-repellent is not particularly limited and can be appropriately selected depending on the purpose thereof, and examples thereof include a method of forming a fluorine-based resin coating film, a fluorine plasma treatment, and emboss processing. In particular, a decrease in the reagent due to spillage of the liquid, the uncertainty, and an increase in the coefficient of variation can be suppressed by performing a water-repellent treatment that provides a contact angle of 1000 or greater.

[Base Material]

The container is preferably a plate-like container in which wells are provided on a base material, but may be a connection type well tube such as an octet tube. The material, the shape, the size, the structure, and the like of the base material are not particularly limited and can be appropriately selected depending on the purpose thereof.

The material of the base material is not particularly limited and can be appropriately selected depending on the intended purpose thereof, and examples thereof include semiconductors, ceramics, metals, glass, quartz glass, and plastics. Among these, plastics are preferable.

Examples of the plastics include polystyrene, polypropylene, polyethylene, a fluororesin, an acrylic resin, polycarbonate, polyurethane, polyvinyl chloride, and polyethylene terephthalate.

The shape of the base material is not particularly limited and can be appropriately selected depending on the purpose thereof, and examples thereof include a plate shape. The structure of the base material is not particularly limited and can be appropriately selected depending on the purpose thereof, and examples thereof include a single layer structure or a multi-layer structure.

[Identification Means]

The container may have identification means for identifying information related to specific copy numbers of the target nucleic acids (such as the number of cells). The identification means is not particularly limited and can be appropriately selected depending on the purpose thereof, and examples thereof include a memory, an IC chip, a barcode, a QR code (registered trademark), a radio-frequency identifier (hereinafter, also referred to as an “RFID”), color coding, and printing.

The positions where the identification means is provided and the number of identification means are not particularly limited, and can be appropriately selected depending on the purpose thereof.

The information to be stored in the identification means includes, in addition to information related to the specific copy numbers of the target nucleic acids, for example, analysis results (such as PM values and variations in PM values), cell life and death, information on which of a plurality of wells is filled with the target nucleic acids, the kind of target nucleic acid, the date and time of measurement, and the name of the measurer.

Information stored in the identification means can be read using various reading means, and for example, a barcode reader is used as the reading means in a case where the identification means is a barcode.

A method of writing information in the identification means is not particularly limited and can be appropriately selected depending on the purpose thereof, and examples thereof include manual input, a method of writing data directly from a liquid droplet-forming device that counts the specific copy number of the target nucleic acids in a case of dispensing the target nucleic acids into wells, transfer of data stored in a server, and transfer of data stored in the cloud.

[Other Members]

Other members are not particularly limited and can be appropriately selected depending on the purpose thereof, and examples thereof include a sealing member.

It is preferable that the container have a sealing member in order to prevent foreign matter from entering the wells or prevent a filling material from flowing out. The sealing member may be configured to be separable along a perforated line such that at least one well can be sealed and each well can be individually sealed or opened.

Examples of the shape of the sealing member include a cap shape that matches the inner wall diameter of the well and a film shape that covers the well opening portion.

Examples of the material for the sealing member include a polyolefin resin, a polyester resin, a polystyrene resin, and a polyamide resin. It is preferable that the sealing member have a film shape that enables sealing of all the wells at once. Further, the sealing member may be configured such that the adhesive strength of the wells that require re-opening and the adhesive strength of the wells that do not require re-opening are different from each other to reduce misuse by the user.

<Method of Producing Device>

A method of producing a device includes an incorporation step of incorporating a specific copy number of target nucleic acids into a nucleic acid in the nucleus of a cell, and a target nucleic acid-filling step of filling a specific number of cells by preparing liquid droplets each containing one cell in which the specific copy number of target nucleic acids are incorporated into a nucleic acid in the nucleus and controlling the number of liquid droplets.

The method of producing a device includes preferably a cell suspension generation step and a cell number-counting step and more preferably a step of calculating the certainty of the copy number of the target nucleic acids present in the well estimated in the cell suspension generation step, the target nucleic acid-filling step, and the cell number-counting step, and an output step and a recording step, and further includes other steps as necessary.

[Incorporation Step]

In the incorporation step, a specific copy number of the target nucleic acids are incorporated into the nucleic acid in the nucleus of a cell.

The copy number of the target nucleic acids to be incorporated into the nucleic acid in the nucleus of a cell is not particularly limited as long as the copy number of a specific copy number, but is preferably one copy from the viewpoint of efficiency of introduction.

Examples of the method of incorporating the target nucleic acids into the nucleic acid in the nucleus of a cell include the same methods exemplified as “method of gene introduction” in “target nucleic acid” described above.

[Target Nucleic Acid-Filling Step]

In the target nucleic acid-filling step, a specific number of cells are filled by preparing liquid droplets each containing one cell in which the specific copy number of target nucleic acids are incorporated into the nucleic acid in the nucleus and controlling the number of liquid droplets.

The preparation and filling of the liquid droplets can be achieved, for example, by jetting a cell suspension containing cells in which the target nucleic acids have been incorporated into the nucleic acid in the nucleus, as liquid droplets, to allow the liquid droplets to sequentially land in the container. The term “jetting” denotes flying of the cell suspension as liquid droplets. The term “sequentially” denotes in order. The term “landing” denotes allowing the liquid droplets to reach the well.

As the container, it is preferable to use a one-hole microtube, an octet tube, a 96-hole well plate, a 384-hole well plate, or the like. In a case where a plurality of wells are present, the same number of cells can be dispensed into wells in these plates or different numbers of cells can also be placed. Further, wells containing no cells may also be present.

As the jetting means, means for jetting the cell suspension as liquid droplets (hereinafter, also referred to as “jet head”) can be suitably used.

Examples of a method of jetting the cell suspension as liquid droplets include an on-demand method and a continuous method in an ink jet method. Among these, in a case of the continuous method, the dead volume of the cell suspension to be used is likely to increase because of reasons such as idle jetting until the jetting state is stabilized, adjustment of the amount of liquid droplets, and continuous formation of liquid droplets even in a case of movement between wells. In the present embodiment, it is preferable to reduce the influence of the dead volume from the viewpoint of adjusting the number of cells. Therefore, the on-demand method is more suitable between the above-described two methods.

Examples of the on-demand method include a plurality of known methods such as a pressure application method of applying a pressure to a liquid to jet the liquid, a thermal method of carrying out film boiling by heating to jet a liquid, and an electrostatic method of pulling liquid droplets using an electrostatic attractive force to form liquid droplets. Among these, the pressure application method is preferable for the following reasons.

In the electrostatic method, it is necessary to provide an electrode to face a jetting unit that holds the cell suspension and forms liquid droplets. In the method of producing a device, it is preferable that an electrode not be disposed from the viewpoint of increasing the degree of freedom of the plate configuration because plates for receiving liquid droplets are disposed to face each other. In the thermal method, since heating occurs locally, there are concerns of the influence of heating on the cells, which are biomaterials, and burning (kogation) of a heater portion. Since the influence of heat depends on the contents and the applications of the plate, it is not necessary to exclude all the factors causing the problem, but the pressure application method is preferable from the viewpoint that there is no concern of burning of the heater portion due to the thermal method.

Examples of the pressure application method include a method of applying a pressure to a liquid using a piezo element and a method of applying a pressure using a valve such as an electromagnetic valve. FIGS. 3 to 5 show configuration examples of liquid droplet generation devices that can be used to jet liquid droplets of a cell suspension.

FIG. 3 is a schematic view showing an example of an electromagnetic valve type jet head. The electromagnetic valve type jet head includes an electric motor 13a, an electromagnetic valve 112, a liquid chamber 11a, a cell suspension 300a, and a nozzle 111a. As the electromagnetic valve type jet head, for example, a dispenser (manufactured by Techelan LLC.) can be suitably used.

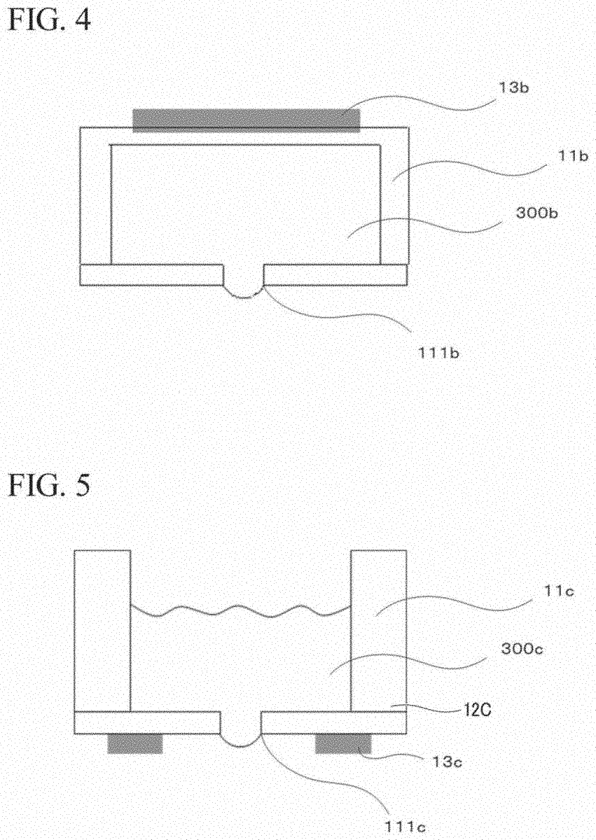

FIG. 4 is a schematic view showing an example of a piezo type jet head. The piezo type jet head includes a piezoelectric element 13b, a liquid chamber 11b, a cell suspension 300b, and a nozzle 111b. A single-cell printer (manufactured by Cytena GmbH) or the like can be suitably used as the piezo type jet head.

Any of these jet heads can be used, but it is preferable to use the piezo type jet head in order to increase the throughput of generation of the plate because liquid droplets cannot be repeatedly formed at a high speed in a case of using the pressure application method with an electromagnetic valve. Further, in the piezo type jet head using a typical piezoelectric element 13b, problems of unevenness in cell concentration due to sedimentation and nozzle clogging may occur.

Therefore, the configuration shown in FIG. 5 and the like is an exemplary example of a more preferable configuration. FIG. 5 is a schematic view showing a modified example of the piezo type jet head formed of the piezoelectric element in FIG. 4. The jet head of FIG. 5 includes a piezoelectric element 13c, a liquid chamber 11c, a cell suspension 300c and a nozzle 111c.

In the jet head of FIG. 5, a compressive stress is applied in the horizontal direction of the paper surface so that a membrane 12c can be deformed in the vertical direction of the paper surface, by applying a voltage to the piezoelectric element 13c from a control device (not shown).

Examples of methods other than the on-demand method include a continuous method of continuously forming liquid droplets. In the continuous method, in a case where liquid droplets are pressurized and pushed out from a nozzle, periodic fluctuations are provided by a piezoelectric element or a heater, and thus minute liquid droplets can be continuously produced. Further, in a case where a voltage is applied to control the jetting direction of the liquid droplets in flight, it is possible to select whether the liquid droplets land on the well or the liquid droplets are collected by a collection unit. Such a method is used for a cell sorter or a flow cytometer, and for example, a cell sorter SH800Z (device name, manufactured by Sony Corporation) can be used.

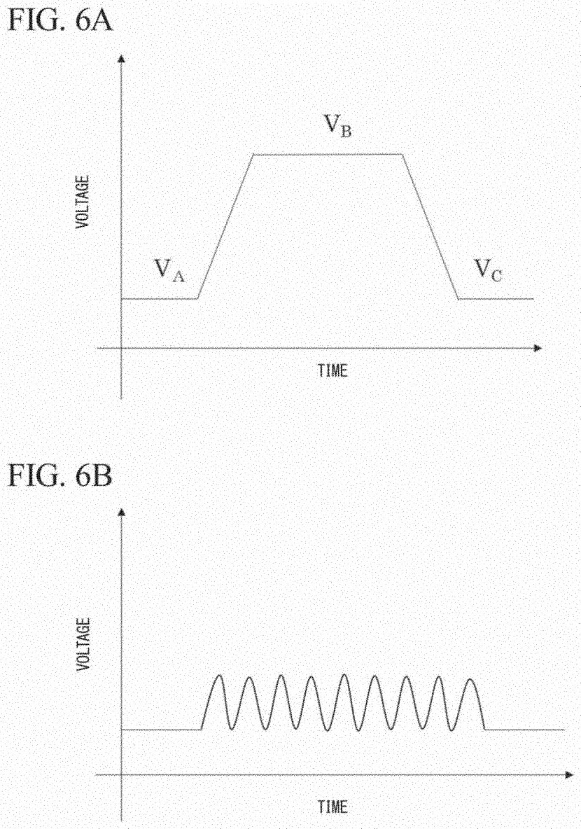

FIG. 6A is a schematic diagram showing an example of a voltage applied to a piezoelectric element. FIG. 6B is a schematic diagram showing another example of a voltage applied to a piezoelectric element. FIG. 6A shows a drive voltage for forming liquid droplets. The liquid droplets can be formed by the magnitude of the voltages (V_{A}, V_{B}, and V_{C}). FIG. 6B shows the voltage for stirring the cell suspension without jetting liquid droplets.

By inputting a plurality of pulses that are not strong enough to jet liquid droplets during the period in which liquid droplets are not jetted, the cell suspension in the liquid chamber can be stirred, and the concentration distribution due to cell sedimentation can be suppressed.

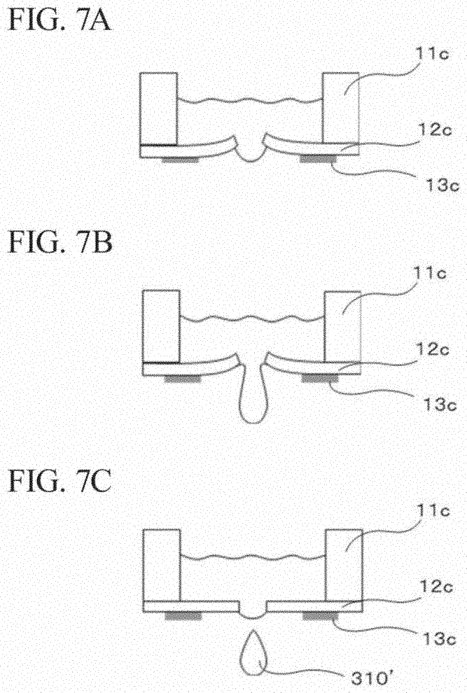

A liquid droplet formation operation of the jet head that can be used in the present embodiment will be described below. The jet head is capable of jetting liquid droplets by applying a pulsed voltage to the upper and lower electrodes formed on the piezoelectric element. FIGS. 7A to 7C are schematic views showing states of liquid droplets at respective timings.

First, as shown in FIG. 7A, since the membrane 12c is drastically deformed by applying a voltage to the piezoelectric element 13c, a high pressure is generated between the cell suspension held in the liquid chamber 11c and the membrane 12c, and thus the liquid droplets are pushed out from the nozzle portion by this pressure.

Next, as shown in FIG. 7B, the liquid droplets are continuously pushed out from the nozzle portion and the liquid droplets grow until the pressure relaxes upward. Finally, as shown in FIG. 7C, in a case where the membrane 12c returns to the original state, the liquid pressure near the interface between the cell suspension and the membrane 12c decreases, and a liquid droplet 310′ is formed.

In the method of producing a device, the liquid droplets may sequentially land on wells by fixing a container consisting of a plate where the wells are formed on a movable stage and combining driving of the stage and formation of the liquid droplets from the jet head. Here, the method of moving the plate is shown as the movement of the stage, but it goes without saying that the jet head may be allowed to move.

The plate is not particularly limited, and a plate where wells are formed, which has been typically used in the field of biotechnology, can be used. The number of wells in the plate is not particularly limited and can be appropriately selected depending on the purpose thereof, and one or a plurality of wells may be present. As the plate, specifically, it is preferable to use a one-hole microtube, an octet tube, a 96-hole well plate, a 384-hole well plate, or the like. In a case where a plurality of wells are present, the same number of cells can be dispensed into wells in these plates or different numbers of cells can also be placed. Further, wells containing no cells may also be present.

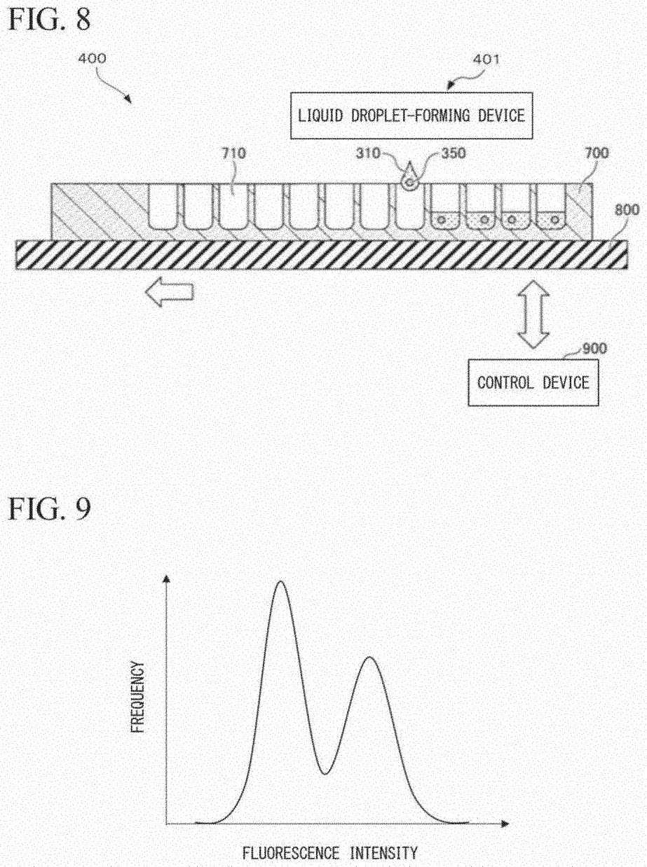

FIG. 8 is a schematic view showing an example of a dispensing device 400 for allowing liquid droplets to sequentially land on the wells of a plate. As shown in FIG. 8, the dispensing device 400 for allowing liquid droplets to land includes a liquid droplet-forming device 401, a plate 700, a stage 800, and a control device 900.

In the dispensing device 400, the plate 700 is disposed on the stage 800 configured to be movable. The plate 700 is formed with a plurality of wells 710 (recessed portions) on which liquid droplets 310 jetted from a jet head of the liquid droplet-forming device 401 land. The control device 900 moves the stage 800 and controls the relative positional relationship between the jet head of the liquid droplet-forming device 401 and each well 710. In this manner, the liquid droplets 310 containing fluorescent-stained cells 350 can be sequentially jetted from the jet head of the liquid droplet-forming device 401 to the respective wells 710.

The control device 900 can be configured to include a CPU, a ROM, a RAM, a main memory, and the like. In this case, various functions of the control device 900 can be realized by causing a main memory to read a program recorded in a ROM or the like and causing the CPU to execute the program. However, a part or the entirety of the control device 900 may be realized only by hardware. Further, the control device 900 may be physically configured of a plurality of devices and the like.

As the liquid droplets to be jetted, it is preferable that the liquid droplets be allowed to land on the wells to obtain a plurality of levels in a case where the cell suspension is allowed to land on the wells. The plurality of levels denote a plurality of standards that serve as a reference. Examples of the plurality of levels include a predetermined concentration gradient of a plurality of cells having a nucleic acid in a well. The plurality of levels can be controlled by using values counted by a sensor.

[Cell Suspension Generation Step]

The cell suspension generation step is a step of generating a cell suspension containing a solvent and a plurality of cells in which the above-described nucleic acid has been introduced into the nucleic acid in the nucleus. The solvent denotes a liquid used to disperse the cells. The suspension in the cell suspension denotes a state where cells are dispersed in the solvent. The generation denotes production.

(Cell Suspension)

The cell suspension contains a solvent and a plurality of cells in which the target nucleic acid has been introduced into the nucleic acid in the nucleus, preferably contains additives, and further contains other components as necessary. The plurality of cells in which the target nucleic acid has been introduced into the nucleic acid in the nucleus are as described in the section of “cell” above.

(Solvent)

The solvent is not particularly limited and can be appropriately selected depending on the purpose thereof, and examples thereof include water, a culture medium, a separate liquid, a diluent, a buffer solution, an organic solution, an organic solvent, a polymer gel solution, a colloidal dispersion liquid, an electrolyte aqueous solution, an inorganic salt aqueous solution, a metal aqueous solution, and a mixed solution thereof. These may be used alone or in combination of two or more kinds thereof. Among these, water and a buffer solution are preferable, and water, a phosphate-buffered saline (PBS) and a Tris-EDTA buffer solution (TE) are more preferable.

(Additive)

The additive is not particularly limited and can be appropriately selected depending on the purpose thereof, and examples thereof include a surfactant, a nucleic acid, and a resin. These may be used alone or in combination of two or more kinds thereof.

The surfactant can prevent aggregation of cells and improve continuous jetting stability. The surfactant is not particularly limited and can be appropriately selected depending on the purpose thereof, and examples thereof include an ionic surfactant and a nonionic surfactant. These may be used alone or in combination of two or more kinds thereof. Among these, a nonionic surfactant is preferred from the viewpoint that proteins are not denatured or deactivated depending on the amount added.

Examples of the ionic surfactant include fatty acid sodium, fatty acid potassium, sodium alpha sulfo fatty acid ester, sodium linear alkyl benzene sulfonate, sodium alkyl sulfate, sodium alkyl ether sulfate, and sodium alpha olefin sulfonate. These may be used alone or in combination of two or more kinds thereof. Among these, fatty acid sodium is preferable, and sodium dodecyl sulfate (SDS) is more preferable.

Examples of the nonionic surfactant include alkyl glycoside, alkyl polyoxyethylene ether (Brij Series or the like), octylphenol ethoxylate (Triton X Series, Igepal CA Series, Nonidet P Series, or Nikkol OP Series), polysorbates (Tween Series such as Tween 20), sorbitan fatty acid ester, polyoxyethylene fatty acid ester, alkyl maltoside, sucrose fatty acid ester, glycoside fatty acid ester, glycerin fatty acid ester, propylene glycol fatty acid ester, and fatty acid monoglyceride. These may be used alone or in combination of two or more kinds thereof. Among these, polysorbates are preferable.

The content of the surfactant is not particularly limited and can be appropriately selected depending on the purpose thereof, but is preferably 0.001% by mass or greater and 30% by mass or less with respect to the total amount of the cell suspension. Since the effect of addition of the surfactant can be obtained in a case where the content is 0.001% by mass or greater, and aggregation of cells can be suppressed in a case where the content thereof is 30% by mass or less, the copy number of nucleic acids in the cell suspension can be strictly controlled.

The nucleic acid is not particularly limited as long as the detection of the target nucleic acid contained in a cell is not affected, and can be appropriately selected depending on the purpose thereof, and examples thereof include ColE1 DNA. In a case where the nucleic acid is added, adhesion of the target nucleic acids contained in a cell to the wall surface of the well used in a case of extraction and detection of the target nucleic acids can be prevented.

The resin is not particularly limited and can be appropriately selected depending on the purpose thereof, and examples thereof include polyethyleneimide.

(Other Components)

Other components are not particularly limited and can be appropriately selected depending on the purpose thereof, and examples thereof include a crosslinking agent, a pH adjuster, a preservative, an antioxidant, an osmotic pressure adjuster, a wetting agent, and a dispersant.

A method of dispersing cells is not particularly limited and can be appropriately selected depending on the purpose thereof, and examples thereof include a media method such as a beads mill, an ultrasonic method such as an ultrasonic homogenizer, and a method of using a pressure difference such as a French press. These may be used alone or in combination of two or more kinds thereof. Among these, the ultrasonic method is preferable from the viewpoint of less damage to cells. In the media method, the cell membrane and the cell wall may be destroyed, and the media may be mixed as contaminants due to the strong disintegration ability.

A method of screening cells is not particularly limited and can be appropriately selected depending on the purpose thereof, and examples thereof include wet classification, and screening using a cell sorter or a filter. These may be used alone or in combination of two or more kinds thereof. Among these, screening using a cell sorter or a filter is preferable from the viewpoint of less damage to cells.

It is preferable that the copy number or the number of molecules of the nucleic acids be estimated from the number of cells contained in the cell suspension by measuring the cell cycle of the cells. The measurement of the cell cycle denotes quantification of the number of cells due to cell division. The estimation of the copy number or the number of molecules of the nucleic acids denotes acquirement of the copy number or the number of molecules of the nucleic acids from the number of cells.

The object to be counted may be the number of nucleic acids contained instead of the number of cells. Typically, since introduction of one copy or one molecule of the target nucleic acid into each cell is selected, or the nucleic acid is introduced into a cell by gene recombination, the number of molecules of the nucleic acids is considered to be the same as the number of cells. However, since cells undergo cell division in a specific cycle, replication of nucleic acids is carried out in the cells. The cell cycle varies depending on the kind of cell, but the expected value and the certainty of the number of molecules of the nucleic acids contained in one cell can be calculated by extracting a predetermined amount of solution from the cell suspension and measuring the cycle of a plurality of cells. In this manner, for example, nuclear-stained cells can be observed with a flow cytometer.

The certainty denotes the probability of the occurrence of a specific event by predicting in advance the degree of possibility of the occurrence of one particular event in a case where there is a possibility of the occurrence of several events. The calculation denotes that a numerical value is acquired by calculation.

FIG. 9 is a graph showing an example of a relationship between the frequency of DNA-replicated cells and the fluorescence intensity. As shown in FIG. 9, two peaks appear on the histogram depending on the presence or absence of DNA replication, and thus the percentage of existing DNA-replicated cells can be calculated. The average copy number or the average number of molecules of nucleic acids contained in one cell can be calculated based on the calculation results, and the estimated copy number or the estimated number of molecules of the nucleic acids can be calculated by multiplying the calculated value by the cell counted results.

Further, it is preferable to perform a treatment of controlling the cell cycle before preparation of the cell suspension, and the copy number or the number of molecules of the nucleic acids can be more accurately calculated from the number of cells by aligning the state before or after the above-described replication occurs.

It is preferable to calculate the certainty (probability) of the estimated specific copy number or number of molecules. By calculating the certainty (probability), it is possible to express and output the certainty as dispersion or standard deviation based on these numerical values. In a case where the effects of a plurality of factors are summed, the square root sum of squares of standard deviations which has been typically used can be used. For example, the rate of correct answers for the number of jetted cells, the number of DNAs in cells, the rate of landing of jetted cells on wells, and the like can be used as the factors. It is also possible to select and calculate an item having a large influence from among the above-described factors.

[Cell Number-Counting Step]

The cell number-counting step is a step of counting the number of cells contained in the liquid droplets with a sensor after the preparation of liquid droplets and before the landing of the liquid droplets on the wells. The sensor denotes a device that replaces the mechanical, electromagnetic, thermal, acoustic, or chemical properties of natural phenomena or man-made objects, or the spatial information and time information indicated by these properties, with signals of other media that are easily handled by humans or machines by applying scientific principles. The counting denotes that the number is counted.

The cell number-counting step is not particularly limited as long as the number of cells contained in the liquid droplets is counted by a sensor after the jetting of the liquid droplets and before the landing of the liquid droplets on the wells, and can be appropriately selected depending on the purpose thereof, and examples thereof include observing cells before jetting and counting cells after landing.

It is preferable that the number of cells contained in the liquid droplets be counted after the jetting of the liquid droplets and before the landing of the liquid droplets on the wells by observing the cells in the liquid droplets at the timing at which the liquid droplets are at positions immediately above the well opening portions where the liquid droplets are predicted to reliably enter the wells of the plate.

Examples of the method of observing cells in the liquid droplets include an optical detection method and an electrical or magnetic detection method.

(Method of Optical Detection)

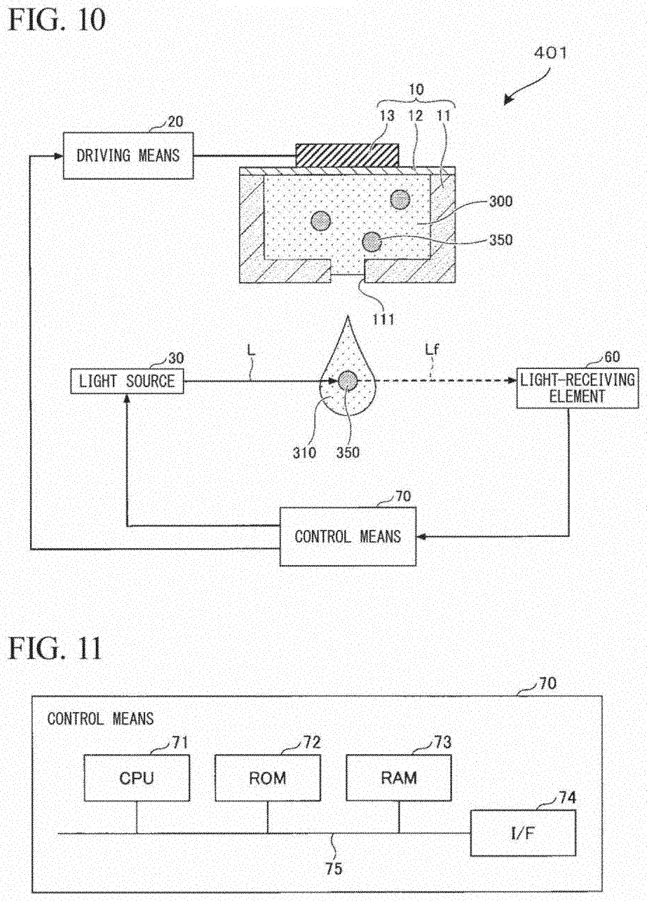

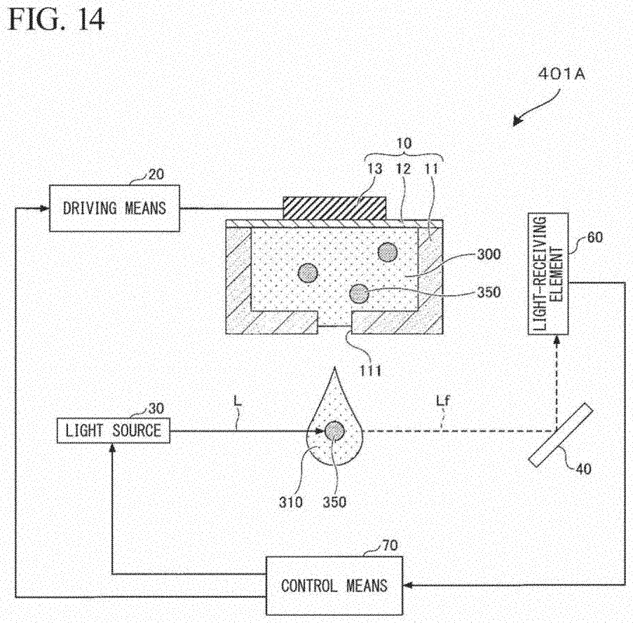

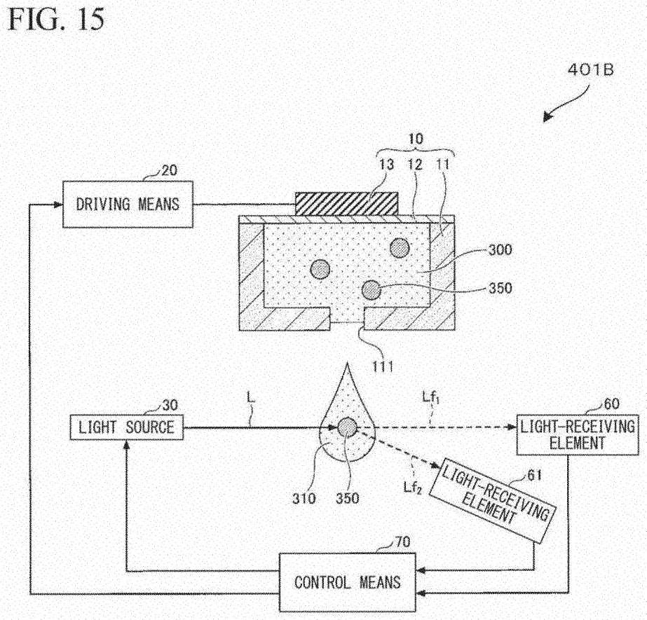

The optical detection method will be described below with reference to FIGS. 10, 14, and 15. FIG. 10 is a schematic view showing an example of the liquid droplet-forming device 401. FIGS. 14 and 15 are schematic views showing other examples (401A, 401B) of the liquid droplet-forming device. As shown in FIG. 10, the liquid droplet-forming device 401 includes a jet head (liquid droplet-jetting means) 10, driving means 20, a light source 30, a light-receiving element 60, and control means 70.

In FIG. 10, a liquid obtained by fluorescently staining cells with a specific dye and dispersing the cells in a predetermined solution is used as a cell suspension, and the number of cells is counted by irradiating liquid droplets formed from the jet head with light having a specific wavelength emitted from the light source and detecting the fluorescence emitted from the cells with the light-receiving element. Here, in addition to the method of staining the cells with a fluorescent dye, the autofluorescence emitted by the molecules originally contained in the cells may be used, or genes encoding fluorescent proteins (for example, green fluorescent protein (GFP)) may be introduced in advance into the cells so that the cells emit fluorescence. The irradiation with light denotes that light is applied.

A jet head 10 includes a liquid chamber 11, a membrane 12, and a driving element 13 and is capable of jetting a cell suspension 300 in which fluorescent-stained cells 350 are suspended as liquid droplets.

The liquid chamber 11 is a liquid-holding portion that holds the cell suspension 300 in which the fluorescent-stained cells 350 are suspended, and a nozzle 111 that is a through-hole is formed on a lower surface side. The liquid chamber 11 can be formed of a metal, silicon, ceramics, or the like. Examples of the fluorescent-stained cells 350 include inorganic fine particles and organic polymer particles that have been stained with a fluorescent dye.

The membrane 12 is a film-like member fixed to an upper end portion of the liquid chamber 11. The planar shape of the membrane 12 can be, for example, circular, but may be elliptical, rectangular, or the like.

The driving element 13 is provided on an upper surface side of the membrane 12. The shape of the driving element 13 can be designed according to the shape of the membrane 12. For example, in a case where the planar shape of the membrane 12 is circular, it is preferable that a circular driving element 13 be provided.

The membrane 12 can be vibrated by supplying a driving signal from the driving means 20 to the driving element 13. The liquid droplets 310 containing the fluorescent-stained cells 350 can be jetted from the nozzle 111 by vibrating the membrane 12.

In a case where a piezoelectric element is used as the driving element 13, for example, a structure in which electrodes for applying a voltage are provided on the upper surface and the lower surface of the piezoelectric material can be employed. In this case, a compressive stress is applied in the horizontal direction of the paper surface by applying a voltage between the upper and lower electrodes of the piezoelectric element from the driving means 20 so that the membrane 12 can be vibrated in the vertical direction of the paper surface. For example, lead zirconate titanate (PZT) can be used as the piezoelectric material. Various other piezoelectric materials such as a bismuth iron oxide, a metal niobate, barium titanate, and any of these materials to which metals or different oxides have been added can be used.

The light source 30 irradiates the liquid droplets 310 in flight with light L. Further, “in flight” denotes a state of the liquid droplets 310 after being jetted from the liquid droplet-jetting means 10 until the liquid droplets land on the object. The liquid droplet 310 in flight has a substantially spherical shape at the position irradiated with the light L. Further, the beam shape of the light L is substantially circular.

Here, it is preferable that the beam diameter of the light L be approximately 10 to 100 times the diameter of the liquid droplet 310. The reason for this is that the liquid droplets 310 are reliably irradiated with the light L from the light source 30 even in a case where variations in positions of the liquid droplets 310 are present.

However, it is not preferable that the beam diameter of the light L greatly exceed 100 times the diameter of the liquid droplet 310. The reason for this is that the energy density of the light to be applied to the liquid droplet 310 decreases, and thus the light amount of fluorescence Lf that emits the light L as excitation light decreases and the light is unlikely to be detected by the light-receiving element 60.

It is preferable that the light L emitted from the light source 30 be pulsed light, and suitable examples thereof include a solid-state laser, a semiconductor laser, and a dye laser. The pulse width of the light L in a case where the light L is pulsed light is preferably 10 μs or less and more preferably 1 μs or less. The energy per unit pulse greatly depends on the optical system such as the presence or absence of light condensing, but the energy thereof is preferably approximately 0.1 μJ or greater and more preferably 1 μJ or greater.

The light-receiving element 60 receives the fluorescence Lf emitted by the fluorescent-stained cells 350 absorbing the light L as excitation light in a case where the liquid droplets 310 in flight contain the fluorescent-stained cells 350. Since the fluorescence Lf is emitted in all directions from the fluorescent-stained cells 350, the light-receiving element 60 can be disposed at any position where the fluorescence Lf can be received. Here, in order to improve the contrast, it is preferable that the light-receiving element 60 be disposed at a position where the light L emitted from the light source 30 does not directly enter.

The light-receiving element 60 is not particularly limited as long as the light-receiving element can receive the fluorescence Lf emitted from the fluorescent-stained cells 350, and can be appropriately selected depending on the purpose thereof, and an optical sensor that irradiates liquid droplets with light having a specific wavelength and receives fluorescence from the cells in the liquid droplets is preferable. Examples of the light-receiving element 60 include one-dimensional elements such as a photodiode and a photosensor, but it is preferable to use a photomultiplier tube or an avalanche photodiode in a case where highly sensitive measurement is required. As the light-receiving element 60, for example, a two-dimensional element such as a charge-coupled device (CCD), a complementary-metal-oxide semiconductor (CMOS), or a gate CCD may be used.

Further, since the fluorescence Lf emitted from the fluorescent-stained cells 350 is weaker than the light L emitted from the light source 30, a filter that attenuates the wavelength range of the light L may be provided in front of the light-receiving element 60 (on the side of the light-receiving surface). In this manner, an image of the fluorescent-stained cells 350 with extremely high contrast can be obtained in the light-receiving element 60. As the filter, for example, a notch filter or the like that attenuates a specific wavelength range having the wavelength of the light L can be used.

Further, as described above, it is preferable that the light L emitted from the light source 30 be pulsed light, but light obtained by continuously oscillating the light L emitted from the light source 30 may be used. In this case, it is preferable that the light-receiving element 60 be controlled such that the light-receiving element can receive light at the timing of irradiating the liquid droplets 310 in flight with the continuously oscillated light and the light-receiving element 60 receives the fluorescence Lf.

The control means 70 has a function of controlling the driving means 20 and the light source 30. Further, the control means 70 also has a function of obtaining information based on the amount of light received by the light-receiving element 60 and counting the number of fluorescent-stained cells 350 contained in the liquid droplets 310 (including a case where the number is zero). Hereinafter, the operation of the liquid droplet-forming device 401 having the operation of the control means 70 will be described with reference to FIGS. 11 to 13.

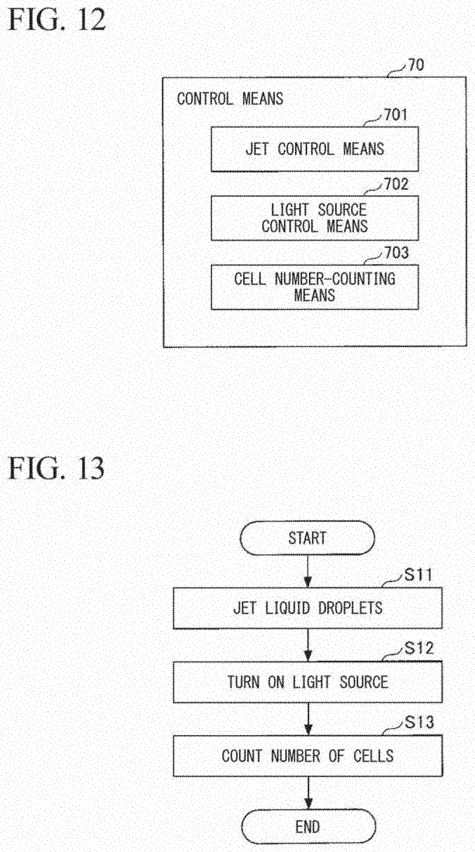

FIG. 11 is a diagram showing hardware blocks of the control means of the liquid droplet-forming device of FIG. 10. FIG. 12 is a diagram showing functional blocks of the control means of the liquid droplet-forming device of FIG. 10. FIG. 13 is a flow chart showing an example of the operation of the liquid droplet-forming device.

As shown in FIG. 11, the control means 70 has a CPU 71, a ROM 72, a RAM 73, a communication interface (communication I/F) 74, and a bus line 75. The CPU 71, the ROM 72, the RAM 73, and the I/F 74 are interconnected via a bus line 75.

The CPU 71 controls each function of the control means 70. The ROM 72, which is a storage means, stores programs executed by the CPU 71 to control each function of the control means 70 and various pieces of information. The RAM 73, which is a storage means, is used as a work area for the CPU 71 and the like. Further, the RAM 73 can temporarily store predetermined information. The IF 74 is an interface for connecting the liquid droplet-forming device 401 to other devices. The liquid droplet-forming device 401 may be connected to an external network or the like via the I/F 74.

As shown in FIG. 12, the control means 70 includes jet control means 701, light source control means 702, and cell number-counting means (cell number-detecting means) 703 as functional blocks.

The cell number counting of the liquid droplet-forming device 401 will be described with reference to FIGS. 12 and 13. First, in Step S11, the jet control means 701 of the control means 70 outputs a command of jetting liquid droplets to the driving means 20. The driving means 20 that has received the command of jetting liquid droplets from the jet control means 701 supplies a driving signal to the driving element 13 to vibrate the membrane 12. The vibration of the membrane 12 causes the liquid droplets 310 containing the fluorescent-stained cells 350 to be jetted from the nozzle 111.

Next, in Step S12, the light source control means 702 of the control means 70 outputs a command of lighting to the light source 30 in synchronization with the jetting of the liquid droplets 310 (in synchronization with the driving signal supplied to the liquid droplet-jetting means 10 from the driving means 20). In this manner, the light source 30 is turned on to irradiate the liquid droplets 310 in flight with the light L.

Here, the synchronization denotes that the light source 30 emits light at the timing at which the liquid droplets 310 are irradiated with the light L in a case where the liquid droplets 310 fly and reach predetermined positions without emitting light at the same time as the liquid droplets 310 are jetted by the liquid droplet-jetting means 10 (at the same time as the driving means 20 supplies the driving signal to the liquid droplet-jetting means 10). That is, the light source control means 702 controls the light source 30 such that the light source 30 emits light with a delay of a predetermined time with respect to the jetting (the driving signal supplied from the driving means 20 to the liquid droplet-jetting means 10) of the liquid droplets 310 by the liquid droplet-jetting means 10.

For example, the velocity v of the liquid droplet 310 to be jetted in a case where the driving signal is supplied to the liquid droplet-jetting means 10 is measured in advance. Further, a time t required for the liquid droplet 310 to reach a predetermined position after being jetted is calculated based on the measured velocity v, and the timing at which the liquid droplet is irradiated with light from the light source 30 is delayed by the time t with respect to the timing at which the driving signal is supplied to the liquid droplet-jetting means 10. In this manner, light emission can be satisfactorily controlled so that the liquid droplets 310 can be reliably irradiated with the light from the light source 30.

Next, in Step S13, the cell number-counting means 703 of the control means 70 counts the number of fluorescent-stained cells 350 contained in the liquid droplets 310 (including a case where the number is zero) based on information from the light-receiving element 60. Here, the information from the light-receiving element 60 is the brightness value (light amount) and the area value of the fluorescent-stained cells 350.

The cell number-counting means 703 can count the number of fluorescent-stained cells 350 by, for example, comparing the amount of light received by the light-receiving element 60 with a preset threshold. In this case, a one-dimensional element or a two-dimensional element may be used as the light-receiving element 60.

In a case where a two-dimensional element is used as the light-receiving element 60, a method of allowing the cell number-counting means 703 to perform image processing for calculating the brightness value or the area of the fluorescent-stained cells 350 based on the two-dimensional image obtained from the light-receiving element 60 may be used. In this case, the cell number-counting means 703 is capable of counting the number of the fluorescent-stained cells 350 by calculating the brightness value or the area value of the fluorescent-stained cells 350 by image processing and comparing the calculated brightness value or the calculated area value with a preset threshold.

Further, the fluorescent-stained cells 350 may be cells or stained cells. A stained cell denotes a cell stained with a fluorescent dye or a cell capable of expressing a fluorescent protein. In the stained cell, the fluorescent dye described above can be used as the fluorescent dye. Further, the fluorescent protein described above can be used as the fluorescent protein.

As described above, in the liquid droplet-forming device 401, the driving signal is supplied from the driving means 20 to the liquid droplet-jetting means 10 holding the cell suspension 300 in which the fluorescent-stained cells 350 are suspended, the liquid droplets 310 containing the fluorescent-stained cells 350 are jetted, and the liquid droplets 310 in flight are irradiated with the light L from the light source 30. Further, the fluorescent-stained cells 350 contained in the liquid droplets 310 in flight emit the fluorescence Lf as excitation light, and the light-receiving element 60 receives the fluorescence Lf. Further, the cell number-counting means 703 counts the number of fluorescent-stained cells 350 contained in the liquid droplets 310 in flight based on the information from the light-receiving element 60.

That is, in the liquid droplet-forming device 401, since the number of fluorescent-stained cells 350 contained in the liquid droplets 310 in flight is actually observed on the spot, the counting accuracy of the number of fluorescent-stained cells 350 can be improved more than the counting accuracy of the related art. In addition, since the fluorescent-stained cells 350 contained in the liquid droplets 310 in flight are irradiated with the light L to emit the fluorescence Lf and the light-receiving element 60 receives the fluorescence Lf, an image of the fluorescent-stained cells 350 can be obtained with high contrast, and the frequency of the occurrence of erroneous counting of the number of fluorescent-stained cells 350 can be reduced.

FIG. 14 is a schematic view showing a modified example of the liquid droplet-forming device 401 of FIG. 10. As shown in FIG. 14, a liquid droplet-forming device 401A is different from the liquid droplet-forming device 401 (see FIG. 10) in that a mirror 40 is disposed in front of the light-receiving element 60. Further, the description of the same components as those of the embodiments described above is not repeated in some cases.

As described above, in the liquid droplet-forming device 401A, the degree of freedom of the layout of the light-receiving element 60 can be improved by disposing the mirror 40 in front of the light-receiving element 60.

For example, there is a concern that interference may occur between the object to land and the optical system (particularly the light-receiving element 60) of the liquid droplet-forming device 401 in the layout of FIG. 10 in a case where the nozzle 111 and the object to land approach each other, but the occurrence of interference can be avoided by employing the layout of FIG. 14.

As shown in FIG. 14, the distance (gap) between the nozzle 111 and the object to land, on which the liquid droplets 310 land, can be decreased by changing the layout of the light-receiving element 60, and thus the variations in landing positions can be suppressed. As a result, the accuracy of dispensing can be improved.

FIG. 15 is a schematic view showing another modified example of the liquid droplet-forming device 401 of FIG. 10. As shown in FIG. 15, the liquid droplet-forming device 401B is different from the liquid droplet-forming device 401 (see FIG. 10) in that a light-receiving element 61 for receiving fluorescence Lf₂ emitted from the fluorescent-stained cells 350 is provided in addition to the light-receiving element 60 for receiving the fluorescence Lf₁ emitted from the fluorescent-stained cells 350. Further, the description of the same components as those of the embodiments described above is not repeated in some cases.

Here, the fluorescence Lf₁ and Lf₂ denote a part of the fluorescence emitted in all directions from the fluorescent-stained cells 350. The light-receiving elements 60 and 61 can be disposed at optional positions where the light-receiving elements can receive the fluorescence emitted from the fluorescent-stained cells 350 in different directions. Further, three or more light-receiving elements may be disposed at positions where the light-receiving elements can receive the fluorescence emitted from the fluorescent-stained cells 350 in different directions. Further, the light-receiving elements may have the same or different specifications from each other.

In a case where the number of light-receiving elements is one, in a case where a plurality of fluorescent-stained cells 350 are contained in the liquid droplet 310 in flight, there is a concern that the number of fluorescent-stained cells 350 contained in the liquid droplet 310 is erroneously counted by the cell number-counting means 703 (counting error occurs) due to overlapping of the fluorescent-stained cells 350.

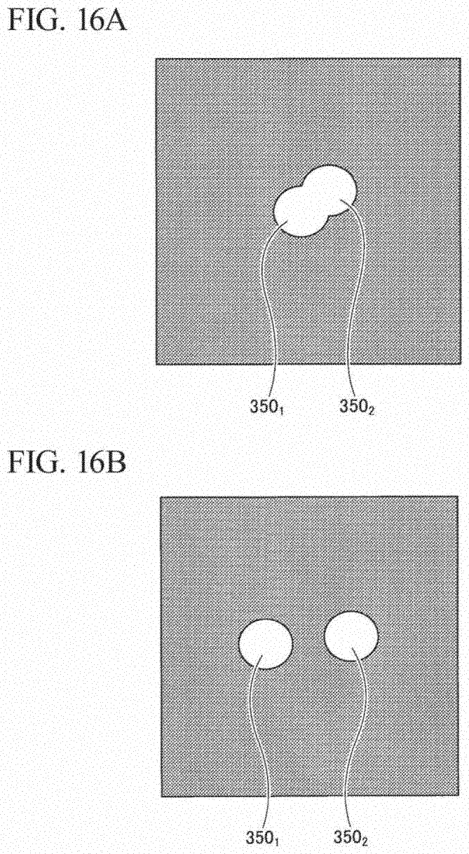

FIGS. 16A and 16B are views showing a case where a liquid droplet in flight contains two fluorescent-stained cells. For example, as shown in FIG. 16A, there may be a case where the fluorescent-stained cells 350a and 350b overlap each other as shown in FIG. 16B or a case where the fluorescent-stained cells 350a and 350b do not overlap each other. The influence of overlapping fluorescent-stained cells can be reduced by providing two or more light-receiving elements.

As described above, the cell number-counting means 703 is capable of counting the number of the fluorescent particles by calculating the brightness value or the area value of the fluorescent particles by image processing and comparing the calculated brightness value or the calculated area value with a preset threshold.

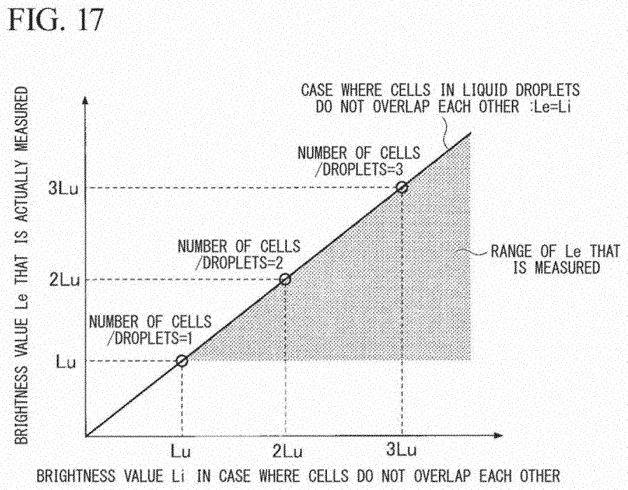

In a case where two or more light-receiving elements are provided, the occurrence of counting errors can be suppressed by adopting data indicating the maximum value among the brightness values or the area values obtained from the respective light-receiving elements. This will be described in more detail with reference to FIG. 17.

FIG. 17 is a diagram showing a relationship between a brightness value Li in a case where particles do not overlap each other and a brightness value Le that is actually measured. As shown in FIG. 17, Le is equal to Li in a case where particles in the liquid droplet do not overlap each other. For example, in a case where the brightness value of one cell is defined as Lu, Le is equal to Lu in a case where “number of cells/droplet=1” is satisfied, and Le is equal to nLu (n represents a natural number) in a case where “number of cells/droplet=n” is satisfied.

However, particles can actually overlap each other in a case where n represents 2 or greater, and thus the actually measured brightness value satisfies “Lu≤Le≤nLu (shaded area in FIGS. 16A & 16B). Therefore, in a case where “number of cells/droplet=n” is satisfied, for example, the threshold can be set as “(nLu−Lu/2)≤threshold <(nLu+Lu/2)”. Further, in a case where a plurality of light-receiving elements are provided, the occurrence of counting errors can be suppressed by adopting data indicating the maximum value among the data obtained from the respective light-receiving elements. Further, the area value may be used instead of the brightness value.

Further, in a case where a plurality of light-receiving elements are provided, the number of cells may be determined by an algorithm for estimating the number of cells based on a plurality of obtained shape data. As described above, since the liquid droplet-forming device 401B has a plurality of light-receiving elements that receive fluorescence emitted from the fluorescent-stained cells 350 in different directions, the frequency of the occurrence of erroneous counting of the number of the fluorescent-stained cells 350 can be further reduced.

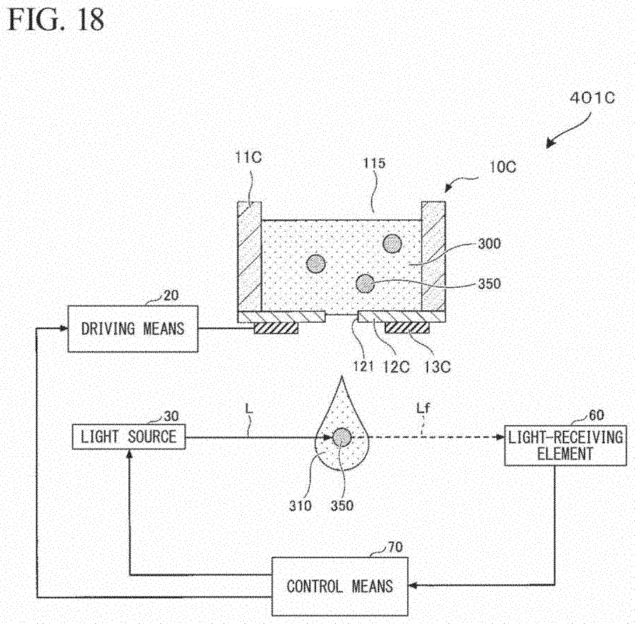

FIG. 18 is a schematic view showing still another modified example of the liquid droplet-forming device 401 of FIG. 10. As shown in FIG. 18, a liquid droplet-forming device 401C is different from the liquid droplet-forming device 401 (see FIG. 10) in that the liquid droplet-jetting means 10 is replaced with a liquid droplet-jetting means 10C. Further, the description of the same components as those of the embodiments described above is not repeated in some cases.

The liquid droplet-jetting means 10C includes a liquid chamber 11C, a membrane 12C, and a driving element 13C. The liquid chamber 11C includes an atmosphere releasing unit 115, which releases the inside of the liquid chamber 11C to the atmosphere, in an upper portion and is configured such that air bubbles mixed into the cell suspension 300 can be discharged from the atmosphere releasing unit 115.

The membrane 12C is a film-like member fixed to a lower end portion of the liquid chamber 11C. A nozzle 121, which is a through-hole, is formed substantially in the center of the membrane 12C, and the cell suspension 300 held in the liquid chamber 11C is jetted as the liquid droplets 310 from the nozzle 121 due to vibration of the membrane 12C. Since the liquid droplets 310 are formed by the inertia of the vibration of the membrane 12C, even the cell suspension 300 with a high surface tension (high viscosity) can be jetted. The planar shape of the membrane 12C can be, for example, circular, but may be elliptical, rectangular, or the like.

The material of the membrane 12C is not particularly limited, but a material with a certain degree of hardness is preferably used from the viewpoint that the membrane 12C easily vibrates in a case where the material is extremely soft, and the vibration is difficult to immediately suppress in a case where liquid droplets are not jetted. As the material of the membrane 12C, for example, a metal material, a ceramic material, a polymer material with a certain degree of hardness, or the like can be used.

Particularly, in a case where cells are used as the fluorescent-stained cells 350, a material having low adhesiveness to cells and proteins is preferable. The adhesiveness of cells is typically considered to depend on the contact angle of the material with water, and the adhesiveness of cells is low in a case where the material is highly hydrophilic or highly hydrophobic. Various metal materials and ceramics (metal oxides) can be used as highly hydrophilic materials, and a fluororesin and the like can be used as highly hydrophobic materials.

Other examples of such materials include stainless steel, nickel, aluminum, silicon dioxide, alumina, and zirconia. In addition to the description above, the cell adhesiveness is considered to be lowered by coating the surface of the material. For example, the surface of the material can be coated with the metal or the metal oxide material described above or with a synthetic phospholipid polymer that imitates a cell membrane (for example, Lipidure, manufactured by NOF Corporation).

It is preferable that the nozzle 121 be formed as a substantially perfect circular through-hole substantially in the center of the membrane 12C. In this case, the diameter of the nozzle 121 is not particularly limited, but it is preferable that the diameter of the nozzle 121 be set to two times or greater the size of the fluorescent-stained cells 350 in order to avoid clogging the nozzle 121 with the fluorescent-stained cells 350. For example, in a case where the fluorescent-stained cells 350 are animal cells, particularly human cells, since the size of human cells is typically approximately in a range of 5 μm to 50 μm, the diameter of the nozzle 121 is set to preferably 10 μm or greater and more preferably 100 μm or greater according to the cells to be used.

Meanwhile, in a case where the liquid droplets are extremely large, since the purpose of forming minute liquid droplets is difficult to achieve, the diameter of the nozzle 121 is preferably 200 μm or less. That is, in the liquid droplet-jetting means 10C, the diameter of the nozzle 121 is typically in a range of 10 μm or greater and 200 μm or less.

The driving element 13 C is formed in a lower surface side of the membrane 12C. The shape of the driving element 13C can be designed according to the shape of the membrane 12C. For example, in a case where the planar shape of the membrane 12C is circular, it is preferable that the driving element 13C having an annular planar shape (ring shape) be formed in the periphery of the nozzle 121. The driving method for the driving element 13C can be set to be the same as the method for the driving element 13.

The driving means 20 is capable of selectively (for example, alternately) applying a jetting waveform for vibrating the membrane 12C to form the liquid droplets 310 and a stirring waveform for vibrating the membrane 12C within a range not forming the liquid droplets 310 to the driving element 13C.

For example, it is possible to prevent the liquid droplets 310 from being formed by applying the stirring waveform by setting both the jetting waveform and the stirring waveform to square waves and setting the drive voltage for the stirring waveform to be lower than the drive voltage for the jetting waveform. That is, the vibration state (degree of vibration) of the membrane 12C can be controlled by changing the magnitude of the drive voltage.

In the liquid droplet-jetting means 10C, since the driving element 13C is formed on a lower surface side of the membrane 12C, the flow of the cell suspension of the liquid chamber 11C can be made in a direction from the lower portion to the upper portion in a case where the membrane 12 is vibrated by the driving element 13C.

Here, the movement of the fluorescent-stained cells 350 is made from the lower portion to the upper portion, and a convection current occurs in the liquid chamber 11C so that the cell suspension 300 containing the fluorescent-stained cells 350 is stirred. Due to the flow of the cell suspension in a direction from the lower portion to the upper portion of the liquid chamber 11C, the sedimented and aggregated fluorescent-stained cells 350 are uniformly dispersed inside the liquid chamber 11C.

That is, the driving means 20 is capable of jetting the cell suspension 300 held in the liquid chamber 11C from the nozzle 121 as the liquid droplets 310 by adding the jetting waveform to the driving element 13C and controlling the vibration state of the membrane 12C. Further, the driving means 20 is capable of stirring the cell suspension 300 held in the liquid chamber 11C by adding the stirring waveform to the driving element 13C and controlling the vibration state of the membrane 12C. Further, the liquid droplets 310 are not jetted from the nozzle 121 during stirring.

As described above, in a case where the cell suspension 3X) is stirred while the liquid droplets 310 are not formed, it is possible to prevent the fluorescent-stained cells 350 from being sedimented and being aggregated on the membrane 12C, and the fluorescent-stained cells 350 can be uniformly dispersed in the cell suspension 300. In this manner, it is possible to suppress clogging of the nozzle 121 and variations in the number of fluorescent-stained cells 350 in the liquid droplets 310 to be jetted. As a result, the cell suspension 30) containing the fluorescent-stained cells 350 can be continuously and stably jetted as the liquid droplets 310 for a long period of time.

Further, in the liquid droplet-forming device 401C, air bubbles may be mixed into the cell suspension 300 in the liquid chamber 11C. Even in this case, since the liquid droplet-forming device 401C is provided with the atmosphere releasing unit 115 above the liquid chamber 11C, air bubbles mixed into the cell suspension 300 can be discharged to the outside air through the atmosphere releasing unit 115. In this manner, the liquid droplets 310 can be continuously and stably formed without discarding a large amount of liquid for discharging air bubbles.

That is, the jetting state is affected in a case where air bubbles are mixed in the vicinity of the nozzle 121 or in a case where a plurality of air bubbles are mixed on the membrane 12C, and thus the mixed air bubbles are required to be discharged in order to stably form liquid droplets for a long time. Typically, air bubbles mixed on the membrane 12C move upward naturally or due to vibration of the membrane 12C, but the mixed air bubbles can be discharged from the atmosphere releasing unit 115 because the liquid chamber 11C is provided with the atmosphere releasing unit 115. Therefore, the occurrence of non-jetting can be prevented even in a case where air bubbles are mixed into the liquid chamber 11C, and thus the liquid droplets 310 can be continuously and stably formed.

Further, the membrane 12C may be vibrated within a range not forming liquid droplets at the timing at which the liquid droplets are not formed to positively move the air bubbles above the liquid chamber 11C.

(Method of Electric or Magnetic Detection)

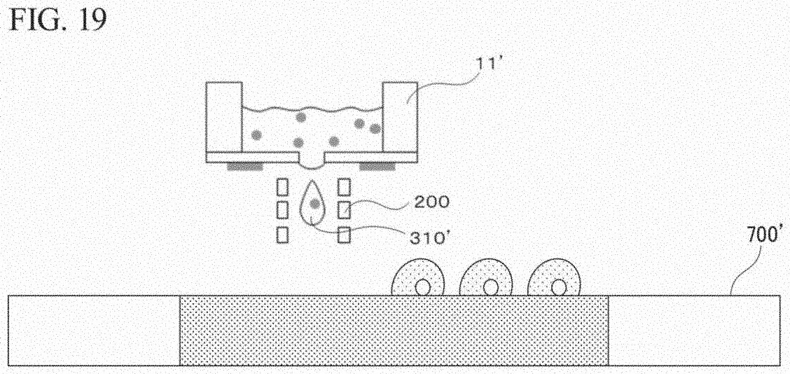

As a method of electric or magnetic detection, a coil 200 for counting the number of cells is provided as a sensor directly below the jet head that jets the cell suspension as liquid droplets 310′ to a plate 700′ from a liquid chamber 11′ as shown in FIG. 19. In a case where cells are covered with magnetic beads that are modified with specific proteins and can adhere to the cells, the presence or absence of cells in the liquid droplets in flight can be detected due to the induced current generated by the cells, to which the magnetic beads have adhered, passing through the coil. In general, since cells have a cell-specific protein on the surface thereof, magnetic beads can adhere to the cells by modifying the magnetic beads with an antibody capable of adhering to the protein. As such magnetic beads, ready-made products can be used. For example, Dynabeads (registered trademark, manufactured by Veritas Corporation) can be used.

(Observation of Cells Before Jetting)

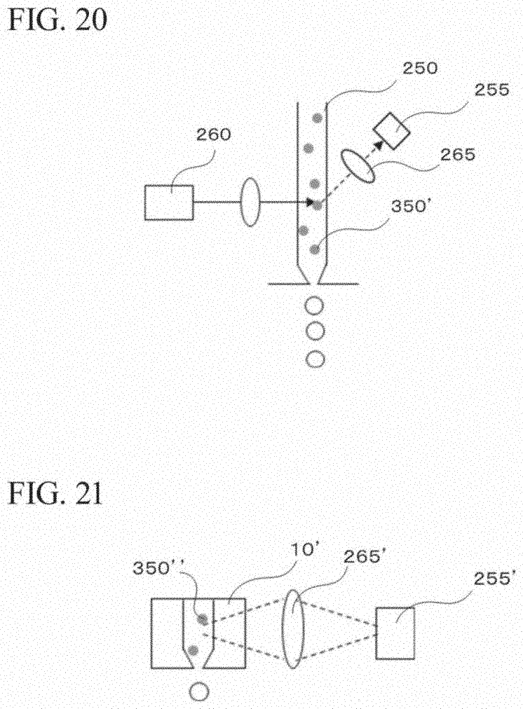

Examples of observing cells before being jetted include a method of counting cells 350′ passing through a microchannel 250 shown in FIG. 20 and a method of acquiring an image in the vicinity of a nozzle portion of a jet head shown in FIG. 21.

The method shown in FIG. 20 is a method used for a cell sorter device, and for example, a cell sorter SH800Z (manufactured by Sony Corporation) can be used. In FIG. 20, liquid droplets can be formed while the presence or absence of cells and the kind of cells are identified by irradiating the microchannel 250 with laser light from a light source 260 and detecting scattered light and fluorescence with a detector 255 using a condenser lens 265. By using the present method, the number of cells that have landed on a predetermined well can be estimated from the number of cells that have passed through the microchannel 250.

A single-cell printer (manufactured by Cytena GmbH) can be used as a jet head 10′ shown in FIG. 21. In FIG. 21, the number of cells having landed on a predetermined well can be estimated before jetting by estimating that cells 350″ in the vicinity of the nozzle portion have been jetted via a lens 265′ based on the results of image acquisition by an image acquisition unit 255′ and estimating the number of cells considered to be jetted due to a difference in the image before and after the jetting. The liquid droplets are continuously generated in the method of counting cells that have passed through the microchannel shown in FIG. 20, whereas the liquid droplets can be formed on demand in FIG. 21, which is more preferable.

(Counting of Cells after Landing)

As a method of counting cells after landing, it is possible to employ a method of detecting fluorescent-stained cells by observing wells in a plate with a fluorescence microscope or the like. This method is described, for example, in Reference Document 1 (Moon S., et al., Drop-on-demand single cell isolation and total RNA analysis, PLoS One, Vol. 6, Issue 3, e17455, 2011).

The method of observing cells before jetting of the liquid droplets and after landing of the liquid droplets has the following problems, and it is most preferable to observe the cells in the liquid droplets being jetted depending on the kind of plate to be generated.

In the method of observing cells before jetting, since the number of cells considered to have landed is counted based on the number of cells that have passed through the channel and the image observation before (and after) jetting, unexpected errors may occur without confirming whether the cells are actually jetted. For example, in a case where the nozzle portion is contaminated, a case where liquid droplets are not correctly jetted and adhere to the nozzle plate and thus the cells in the liquid droplets do not land occurs. In addition, problems such as cells remaining in a narrow region of the nozzle portion and cells moving more than expected due to the jetting operation and leaving the observation range may occur.

Further, the method of detecting cells having landed on the wells also has a problem. First, it is necessary to prepare a plate that enables microscopic observation. In general, plates with transparent and flat bottoms, particularly plates with glass bottoms are used as the plates that enable observation, but these plates are special plates, and thus there is a problem in that typical wells cannot be used. Further, in a case where the number of cells is large, such as several tens of cells, there is a problem in that the cells overlap each other and thus the number of cells cannot be accurately counted.

Therefore, it is preferable that observation of the cells before jetting and counting of the number of cells after landing be performed in addition to counting the number of cells contained in the liquid droplets by a sensor and the cell number-counting means after jetting of the liquid droplets and before landing of the liquid droplets on the wells.

As the light-receiving element, a light-receiving element having one or a small number of light-receiving units, such as a photodiode, an avalanche photodiode, or a photomultiplier tube can be used, or a two-dimensional sensor such as a charge-coupled device (CCD) provided with light-receiving elements in the form of a two-dimensional array, a complementary-metal-oxide semiconductor (CMOS), a gate CCD, or the like can be used.

In a case of using a light-receiving element having one or a small number of light-receiving units, determination of how many cells are contained based on the fluorescence intensity is also considered to be made using a calibration curve prepared in advance, but binary detection of the presence or absence of cells in the liquid droplets in flight is mainly performed. In a case where the cell concentration of the cell suspension is sufficiently low and liquid droplets are jetted in a state where the liquid droplets each contain only 1 or 0 cells, the number of cells can be sufficiently accurately counted by binary detection.

Assuming that the cells are randomly disposed in the cell suspension, the number of cells in the liquid droplets in flight is considered to follow the Poisson distribution, and the probability P (≥2) that the liquid droplets each contain two or more cells is represented by Equation 4.

P(≥2)=1−(1+λ)×e^{-λ} (Equation 4)

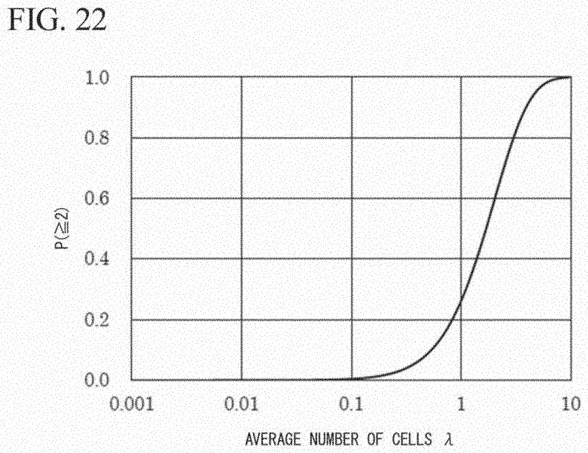

FIG. 22 is a graph showing a relationship between the probability P (≥2) and the average number of cells. Here, k represents the average number of cells in the liquid droplets, which is obtained by multiplying the cell concentration in the cell suspension by the volume of the jetted liquid droplets.

In a case where the number of cells is counted by binary detection, it is preferable that the probability P (≥2) be a sufficiently small value from the viewpoint of ensuring the accuracy and that λ<0.15 in which the probability P (≥2) is 1% or less is satisfied. The light source is not particularly limited as long as the fluorescence of cells can be excited and can be appropriately selected depending on the purpose thereof, and a light source in which a typical lamp such as a mercury lamp or a halogen lamp is provided with a filter to apply light with a specific wavelength to the lamp, a light-emitting diode (LED), or a laser can be used as the light source. However, particularly in a case where minute liquid droplets with a volume of 1 nL or less are formed, it is preferable to use a laser because a narrow region is required to be irradiated with light having high intensity. As a laser light source, various commonly known lasers such as a solid-state laser, a gas laser, and a semiconductor laser can be used. Further, the excitation light source may continuously irradiate a region through which the liquid droplets pass or may irradiate a region in the pulse form at the timing delayed by a predetermined time with respect to the liquid droplet-jetting operation in synchronization with the jetting of the liquid droplets.

[Step of Calculating Certainty of Copy Number of Nucleic Acids Estimated in Cell Suspension Generation Step, Nucleic Acid-Filling Step, and Cell Number-Counting Step]

The present step is a step of calculating the certainty in each step of the cell suspension generation step, the target nucleic acid-filling step, and the cell number-counting step. The certainty of the copy number of target nucleic acids to be estimated can be calculated in the same manner as the certainty in the cell suspension generation step.

Further, as the calculation timing of the certainty, the certainty may be calculated collectively in the step following the cell number-counting step, or the certainty may be calculated at the end of each step of the cell suspension generation step, the target nucleic acid-filling step, and the cell number-counting step, and each certainty may be combined and calculated in the step following the cell number-counting step. In other words, the certainty in each step may be appropriately calculated until the combined calculation.

[Output Step]

The output step is a step of outputting the value counted by the cell number-counting means based on the detection result measured by the sensor as the number of cells contained in the cell suspension that has landed in the container. The counted value denotes the number of cells contained in the container which is counted by the cell number-counting means based on the detection result measured by the sensor.

The output denotes transmission of counted values to a server serving as external counting result storage means as electronic information after a device such as a prime mover, a communication device, or a calculator or printing of counted values as a printed material.

In the output step, the number of cells or the number of target nucleic acids in each well of the plate is observed or estimated in a case of generation of the plate, and the observed value or estimated value is output to an external storage unit. The output may be performed simultaneously with the cell number-counting step or may be performed after the cell number-counting step.

[Recording Step]

The recording step is a step of recording the output observed value or estimated value in the output step. The recording step can be suitably carried out in the recording unit. The recording may be performed simultaneously with the output step or may be performed after the output step. The recording includes not only providing information for a recording medium, but also storing information in a recording unit. In this case, the recording unit can also be referred to as a storage unit.

[Other Steps]

Other steps are not particularly limited and can be appropriately selected depending on the purpose thereof, and examples thereof include an enzyme deactivation step.

The enzyme deactivation step is a step of deactivating an enzyme. Examples of the enzyme include DNase and RNase. The method of deactivating the enzyme is not particularly limited and can be appropriately selected depending on the purpose thereof, and known methods can be suitably used.

### EXAMPLES

Hereinafter, the present invention will be described based on examples, but the present invention is not limited to the following examples.

### Experimental Example 1

The limit of detection of a reagent was examined using commercially available target nucleic acids and reagents.

First, yeasts incorporating the target nucleic acid were prepared by the method described below. Yeasts into which BCR-ABL1 P210 dsDNA was incorporated were prepared as target nucleic acids and yeasts into which ABL1 dsDNA was incorporated were prepared as endogenous control genes.

(Preparation of Gene-Recombination Yeast)

First, budding yeast YIL015W BY4741 (manufactured by ATCC, ATCC4001408) was used as a carrier cell for one molecule of a target nucleic acid to prepare a recombinant. A plasmid introduced to arrange target DNA or endogenous control genes and URA3 genes as a selection marker in tandem was prepared in advance, and homologous recombination was performed using the plasmid so that one molecule of target dsDNA or endogenous control genes were introduced into the Bart gene region of the genomic DNA of the carrier cell, thereby preparing gene-recombination yeasts.

(Culture of Gene-Recombination Yeast and Control of Cell Cycle)

Thereafter, 900 μL of Dulbecco's phosphate-buffered saline (14190-144, manufactured by Thermo Fisher Scientific Co., Ltd., hereinafter, also referred to as “DPBS”) containing 500 μg/mL al-Mating Factor acetate salt (T6901-5MG, manufactured by Sigma-Aldrich CO., LLC, hereinafter, also referred to as “a factor”) was added to an Erlenmeyer flask in which 90 mL of gene-recombination yeast cultured in a 50 g/L YPD culture medium (CLN-630409, manufactured by Takara Bio Inc.) was fractionated, and the mixture was incubated at a temperature of 28° C., and a shaking speed of 250 rpm for 2 hours using a bioshaker (BR-23FH, manufactured by Taitec Co., Ltd.), thereby obtaining a yeast suspension in which the yeasts were synchronized with the G0/G1 phase.

(Immobilization of Gene-Recombination Yeast)

45 mL of the yeast suspension confirmed as having yeasts synchronized with the G0/G1 phase was transferred to a centrifuge tube (VIO-50R, manufactured by AS ONE Corporation), the yeast suspension was centrifuged at a rotation speed of 3000 rpm for 5 minute using a centrifuge (F16RN, manufactured by Hitachi, Ltd.), and the supernatant was removed, thereby obtaining a yeast pellet, 4 mL of formalin (062-01661, manufactured by FUJIFILM Wako Pure Chemical Corporation) was added to the obtained yeast pellets, the mixture was allowed to stand for 5 minutes and centrifuged, and the supernatant was removed to obtain yeast pellets. Next, 10 mL of ethanol was added to the obtained yeast pellets for suspension, thereby obtaining an immobilized yeast suspension.

(Nuclear Staining of Gene-Recombination Yeast)

200 μL of the immobilized yeast suspension was fractionated, washed once with DPBS, and resuspended in 480 μL of DPBS. After 20 μL of 20 mg/mL RNase A (manufactured by Nippon Gene Co., Ltd., 318-06391) was added thereto, the suspension was incubated at 37° C. for 2 hours using a bioshaker. Thereafter, 25 μL of 20 mg/mL proteinase K (TKR-9034, manufactured by Takara Bio Inc.) was added thereto, and the suspension was incubated at 50° C. for 2 hours using Petit Cool (Petit Cool MiniT-C, manufactured by WAKENBTECH Co., Ltd.). Finally, 6 μL of 5 mM SYTOX Green Nucleic Acid Stain (S7020, manufactured by Thermo Fisher Scientific) was added thereto and stained for 30 minutes in a light-shielded environment.

(Dispersion of Gene-Recombination Yeast)

The nuclear-stained yeast suspension was subjected to a dispersion treatment using an ultrasonic homogenizer (LUH150, manufactured by Yamato Scientific Co., Ltd.) at an output of 30% for 10 seconds, thereby obtaining a yeast suspension ink.

(Number Counting Dispensing of Yeast Suspension)

The number of yeast cells in the liquid droplets was counted by the following method, and one cell was jetted to each well.

Specifically, the yeast suspension ink was sequentially jetted at 10 Hz to each well of a 96-well plate (trade name, “MicroAmp™ Fast Optical 96-Well Reaction Plate, 0.1 mL”, manufactured by Thermofisher) using a piezoelectric application type jet head (manufactured by Ricoh Japan Corp.) as the liquid droplet-jetting means and using a liquid droplet-forming device (manufactured by Ricoh Japan Corp.).

A high-sensitivity camera (C13440-20CU, manufactured by Hamamatsu Photonics Co., Ltd.) was used for photographing as light-receiving means for the yeasts in the jetted liquid droplets. AYAG laser (manufactured by Spectra Physics, Explorer ONE-532-200-KE) was used as the light source, and image processing was performed using Image J, which is image-processing software, as particle counting means for the photographed image to count the number of cells. Yeasts containing one molecule of target DNA or endogenous control genes were disposed in each well of the 96-well plates (rows: A to H, columns: 1 to 12) such that the number of dsDNA molecules was as listed in Table 2-1 (ABL1) and Table 2-2 (BCR-ABL1 P210). The 96-well plates listed in Tables 2-1 and 2-2 are the same plates and described separately to facilitate understanding of the disposition of the target DNA and the endogenous control genes. In Table 2-1. “S” represents an RNA extract from a healthy subject used as a negative specimen, and 5 μL of the extract was disposed in each well. Further, two plates with the following disposition were prepared and used for measurement.

TABLE 2-1

| | 1 | 2 | 3 | 4 | 5 | 6 | 7 | 8 | 9 | 10 | 11 | 12 |
|---|---|---|---|---|---|---|---|---|---|---|---|---|
| A | 0 | 0 | 1 | 1 | 10 | 10 | 10² | 10² | 10⁴ | 10⁴ | 10⁶ | 10⁶ |
| B | | | | | | | | | S | S | | |
| C | S | S | S | S | S | S | S | S | S | S | S | S |
| D | S | S | S | S | S | S | S | S | S | S | S | S |
| E | S | S | S | S | S | S | S | S | S | S | S | S |
| F | S | S | S | S | S | S | S | S | S | S | S | S |
| G | S | S | S | S | S | S | S | S | S | S | S | S |
| H | | | | | | | | | | | | |

TABLE 2-2

| | 1 | 2 | 3 | 4 | 5 | 6 | 7 | 8 | 9 | 10 | 11 | 12 |
|---|---|---|---|---|---|---|---|---|---|---|---|---|
| A | 0 | 0 | 1 | 1 | 10 | 10 | 10² | 10² | 10⁴ | 10⁴ | 10⁶ | 10⁶ |
| B | | | | | | | | | | | | |
| C | 0 | 2 | 4 | 8 | 16 | 32 | 0 | 2 | 4 | 8 | 16 | 32 |
| D | 0 | 2 | 4 | 8 | 16 | 32 | 0 | 2 | 4 | 8 | 16 | 32 |
| E | 0 | 2 | 4 | 8 | 16 | 32 | 0 | 2 | 4 | 8 | 16 | 32 |
| F | 0 | 2 | 4 | 8 | 16 | 32 | 0 | 2 | 4 | 8 | 16 | 32 |
| G | 0 | 2 | 4 | 8 | 16 | 32 | 0 | 2 | 4 | 8 | 16 | 32 |
| H | | | | | | | | | | | | |

(Nucleic Acid Extraction)

1 mg/mL of Zymolyase (registered trademark) 100T (07665-55, manufactured by Nacalai Tesque, Inc.) was added to Tris-EDTA (TE) buffer (hereinafter, also referred to as “ColE1/TE”) containing 5 ng/μL of ColE1 DNA (312-00434, manufactured by FUJIFILM Wako Pure Chemical Corporation), thereby preparing a Zymolyase solution.

4 μL of the Zymolyase solution was added to each well of the 96-well plate on which the yeasts were disposed and incubated at 37° C. for 30 minutes to dissolve the cell walls (nucleic acid extraction), and the solution was subjected to a heat treatment at 95° C. for 2 minutes to extract the nucleic acids in the yeast cells.

(Amplification of Target Nucleic Acid by PCR)

Thereafter, the target nucleic acid in each well was amplified under the conditions shown below.

(Conditions for Test)
Composition of reagent: listed in Table 3. Further, “Template” in Table 3 denotes a negative specimen. In the columns 1 to 12 and the row A of the well containing no negative specimen, 5.00 μL of Nuclease-free water was added instead of Template in the composition of Table 3.
PCR device: QuantStudio 12K Flex real-time PCR system
Amplification conditions: listed in Table 4

TABLE 3

| Components | (μL)/well | Conc. |
|---|---|---|
| 2 × Master Mix | 12.50 | |
| RT Enzyme Mix | 1.25 | |
| BCR-ABL1 P210 Forward primer (25 μM) | 0.40 | 400 nM |
| BCR-ABL1 P210 Reverse primer (25 μM) | 0.40 | 400 nM |
| ABL1 Forward primer (25 μM) | 0.40 | 400 nM |
| ABL1 Reverse primer (25 μM) | 0.40 | 400 nM |
| BCR-ABL1 P210 FAM-BHQ1 probe (10 μM) | 0.20 | 80 nM |
| ABL1 HEX-BHQ1 probe (10 μM) | 0.20 | 80 nM |
| Nuclease-free water | 4.25 | |
| Template | 5.00 | |
| Total | 25.00 | |

TABLE 4

| Step | Temperature [° C.] | Duration | | Cycle |
|---|---|---|---|---|
| Reverse Transcription | 55 | 10 | min | 1 |
| Initial Denaturation | 95 | 1 | min | 1 |
| Denaturation | 95 | 10 | sec | 40 |
| Extension | 60 | 1 | min | |

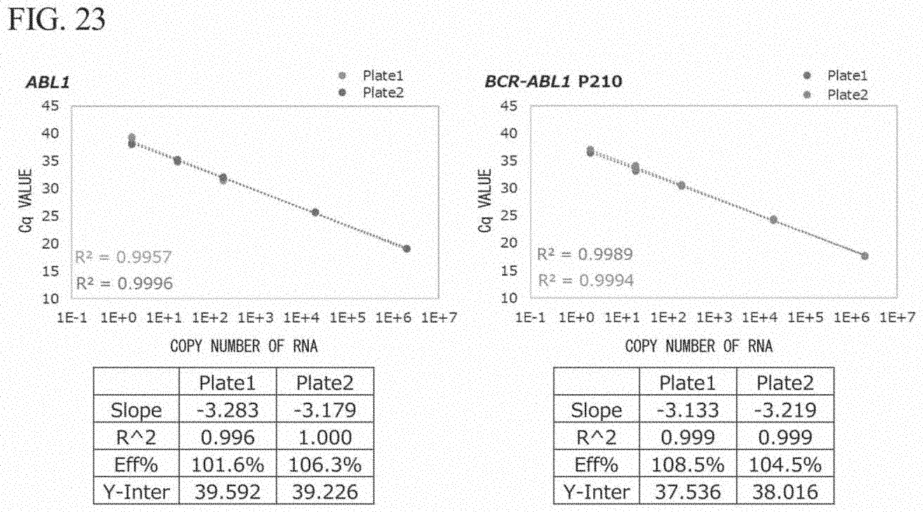

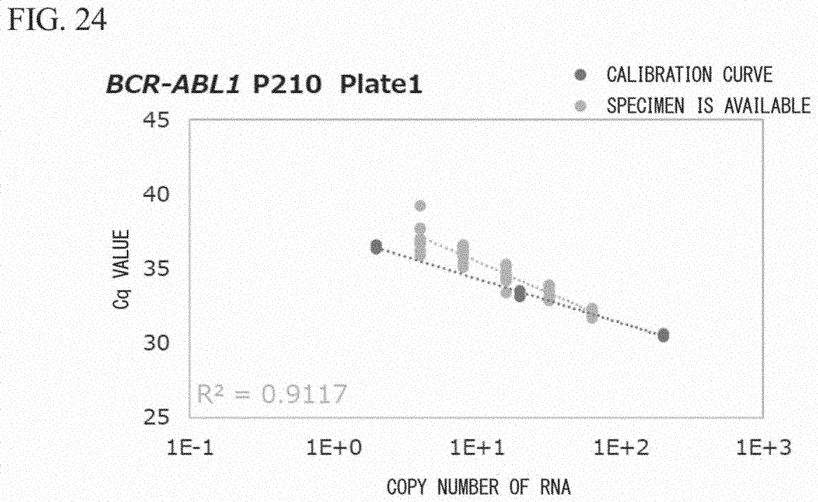

The analysis results after the amplification are listed in Tables 5-1 and 5-2, the graph of the calibration curve of the target nucleic acid and the endogenous control gene is shown in FIG. 23, and the graph of the calibration curve of the target nucleic acid and the measurement results of the positive control group measured in the presence of the negative specimen is shown in FIG. 24. In Tables 5-1 and 5-2, the detection rate was calculated using the formula: {1−(number of UDs)÷(number of times of measurements)}×100 for each copy number. Further, “UD” stands for “Undetermined” (undetected). Further, CVₗₙ was calculated using Equation (I). In Equation (I), E represents an efficiency of a nucleic acid amplification reaction, SD (Cq) represents a standard deviation of Cq, and Cq represents the number of cycles at which an amplification curve and a threshold intersect in the nucleic acid amplification reaction.

CVₗₙ=\sqrt{(1+E)^{(SD)Cq))}^{²}*^{ln(1+E)}−1} Equation (I):

TABLE 5-1

| | Plate 1 copy number of RNA | | | | |
|---|---|---|---|---|---|
| | 4 | 8 | 16 | 32 | 64 |
| Cq Ave | 37.13 | 35.99 | 34.59 | 33.38 | 32.09 |
| σ | 0.99 | 0.49 | 0.58 | 0.41 | 0.19 |
| CV % | 2.67 | 1.36 | 1.68 | 1.22 | 0.58 |
| Detection | 90.0% | 100.0% | 100.0% | 100.0% | 100.0% |
| rate | | | | | |
| CVₗₙ | 58.3% | 27.2% | 32.5% | 22.5% | 10.2% |

TABLE 5-2

| | Plate 2 copy number of RNA | | | | |
|---|---|---|---|---|---|
| | 4 | 8 | 16 | 32 | 64 |
| Cq Ave | 35.49 | 34.60 | 33.65 | 32.74 | 31.85 |
| σ | 0.46 | 0.38 | 0.22 | 0.18 | 0.13 |
| CV % | 1.28 | 1.10 | 0.66 | 0.57 | 0.41 |
| Detection | 100.0% | 100.0% | 100.0% | 100.0% | 100.0% |
| rate | | | | | |
| CVₗₙ | 35.6% | 29.5% | 17.0% | 14.1% | 9.9% |

As shown in FIG. 24, as the copy number of BCR-ABL1 P210 decreased, the Cq value was likely to be deviated from the calibration curve due to the inhibitory effect of the components contained in the negative specimen.

As shown in Tables 5-1 and 5-2, the limit of detection (LOD) was 4 copies of RNA and the limit of quantitation (LOQ) was 8 copies of RNA.

### Experimental Example 2

The limit of detection of a reagent was examined using commercially available target nucleic acids and reagents.

First, yeasts into which the target nucleic acids were incorporated were prepared by the same method as in Experimental Example 1. Yeasts into which BCR-ABL1 P210 dsDNA was incorporated were prepared as target nucleic acids and yeasts into which ABL1 dsDNA was incorporated were prepared as endogenous control genes.

In addition, the same method as in Experimental Example 1 was used from “culture of gene-recombination yeast and control of cell cycle” to “number counting dispensing of yeast suspension”.

Yeasts containing one molecule of target DNA or endogenous control genes were disposed in each well of the 96-well plates (rows: A to H, columns: 1 to 12) such that the number of DNA molecules was as listed in Table 6-1 (ABL1) and Table 6-2 (BCR-ABL1 P210). Further, the 96-well plates listed in Tables 6-1 and 6-2 are the same plate and described separately to facilitate understanding of the disposition of the target DNA and the endogenous control genes. In Table 6-1, “S” represents a cDNA solution from a healthy subject used as a negative specimen, and 10 μL of the RNA extract from a healthy subject was synthesized to cDNA using a reverse transcriptase SuperScript (registered trademark) IV. 5 μL/well of the cDNA solution from a healthy subject was disposed. Further, two plates with the following disposition were prepared and used for measurement.

TABLE 6-1

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | 0 | 0 | 10² | 10² | 10⁴ | 10⁴ | 10⁶ | 10⁶ | S | S | | |
| C | S | S | S | S | S | S | S | S | S | S | S | S |
| D | S | S | S | S | S | S | S | S | S | S | S | S |
| E | S | S | S | S | S | S | S | S | S | S | S | S |
| F | S | S | S | S | S | S | S | S | S | S | S | S |
| G | S | S | S | S | S | S | S | S | S | S | S | S |
| H | | | | | | | | | | | | |

103051

TABLE 6-2

| | 1 | 2 | 3 | 4 | 5 | 6 | 7 | 8 | 9 | 10 | 11 | 12 |
|---|---|---|---|---|---|---|---|---|---|---|---|---|
| A | 0 | 0 | 1 | 1 | 10 | 10 | 10² | 10² | 10⁴ | 10⁴ | 10⁶ | 10⁶ |
| B | | | | | | | | | | | | |
| C | 0 | 2 | 4 | 8 | 16 | 32 | 0 | 2 | 4 | 8 | 16 | 32 |
| D | 0 | 2 | 4 | 8 | 16 | 32 | 0 | 2 | 4 | 8 | 16 | 32 |
| E | 0 | 2 | 4 | 8 | 16 | 32 | 0 | 2 | 4 | 8 | 16 | 32 |
| F | 0 | 2 | 4 | 8 | 16 | 32 | 0 | 2 | 4 | 8 | 16 | 32 |
| G | 0 | 2 | 4 | 8 | 16 | 32 | 0 | 2 | 4 | 8 | 16 | 32 |
| H | | | | | | | | | | | | |

(Nucleic Acid Extraction)

1 mg/mL of Zymolvase (registered trademark) 100T (07665-55, manufactured by Nacalai Tesque, Inc.) was added to Tris-EDTA (TE) buffer (hereinafter, also referred to as “ColE1/TE”) containing 5 ng/μL of ColE1 DNA (312-00434, manufactured by FUJIFILM Wako Pure Chemical Corporation), thereby preparing a Zymolyase solution.

4 μL of the Zymolyase solution was added to each well of the %-well plate on which the yeasts were disposed and incubated at 37° C. for 30 minutes to dissolve the cell walls (nucleic acid extraction), and the solution was subjected to a heat treatment at 95° C. for 2 minutes to extract the nucleic acids in the yeast cells.

(Amplification of Target Nucleic Acid by PCR)

Thereafter, the target nucleic acid in each well was amplified under the conditions shown below.

(Conditions for Test)
Composition of reagent: listed in Table 7-1 (for ABL1) and Table 7-2 (BCR-ABL1 P210). Further, “Template” in Tables 7-1 and 7-2 denotes a negative specimen. The reagents listed in Table 7-1 were disposed in the columns 1 to 10 and the row B in Table 6-1, and the reagents listed in Table 7-2 were disposed in the columns 1 to 12 and the rows C to G in Table 6-2. In the columns 1 to 8 and the row B of the well containing no negative specimen, 5.00 μL of Nuclease-free water was added instead of Template. In the columns 1 to 12 and the row A of the well containing no negative specimen, 5.00 μL of Nuclease-free water was added instead of Template in the composition of Table 2.
PCR device: QuantStudio 12K Flex real-time PCR system
Amplification conditions: listed in Table 8.

TABLE 7-1

| Components | (μL)/well | Conc. |
|---|---|---|
| 2 × Master Mix | 12.50 | |
| ABL1 Forward primer (25 μM) | 0.50 | 500 nM |
| ABL1 Reverse primer (25 μM) | 0.50 | 500 nM |
| ABL1 HEX-BHQ1 probe (10 μM) | 0.50 | 200 nM |
| Nuclease-free water | 6.00 | |
| Template | 5.00 | |
| Total | 25.00 | |

TABLE 7-2

| Components | (μL)/well | Conc. |
|---|---|---|
| 2 × Master Mix | 12.50 | |
| BCR-ABL1 P210 Forward primer (25 μM) | 0.50 | 500 nM |
| BCR-ABL1 P210 Reverse primer (25 μM) | 0.50 | 500 nM |
| BCR-ABL1 P210 FAM-BHQ1 probe (10 μM) | 0.50 | 200 nM |
| Nuclease-free water | 6.00 | |
| Template | 5.00 | |
| Total | 25.00 | |

TABLE 8

| Step | Temperature [° C.] | Duration | | Cycle |
|---|---|---|---|---|
| Polymerase Activation/ | 50 | 2 | min | 1 |
| DNA Denaturation | 95 | 10 | min | 1 |
| Denaturation | 95 | 30 | sec | 40 |
| Extension | 60 | 1 | min | |

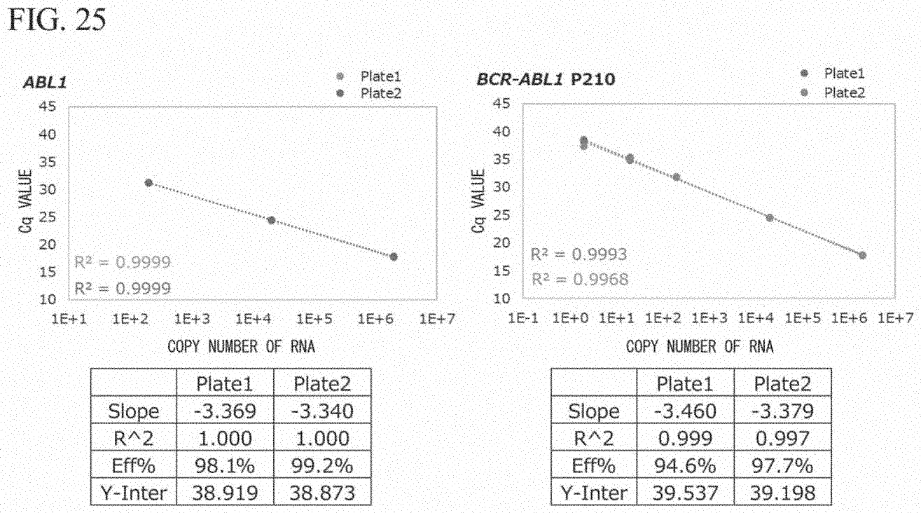

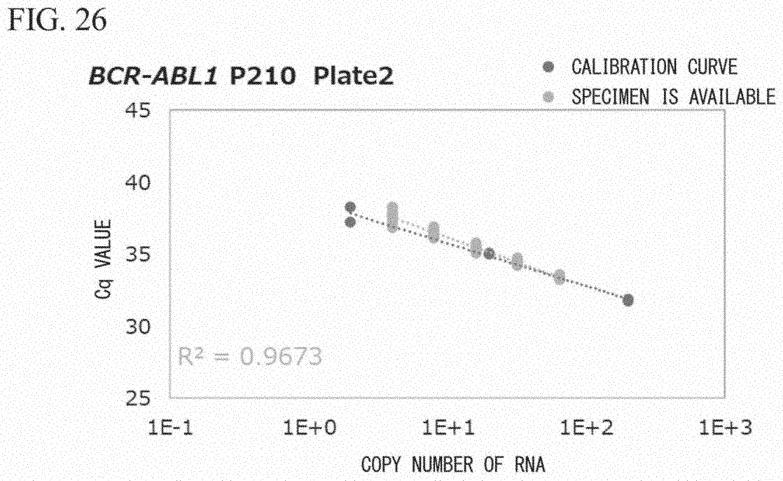

The analysis results after the amplification are listed in Tables 9-1 and 9-2, the graph of the calibration curve of the target nucleic acid and the endogenous control gene is shown in FIG. 25, and the graph of the calibration curve of the target nucleic acid and the measurement results of the positive control group measured in the presence of the negative specimen is shown in FIG. 26. In Tables 9-1 and 9-2, the detection rate was calculated using the formula: {1−(number of UDs)÷(number of times of measurements)}×100 for each copy number. Further, “UD” stands for “Undetermined” (undetected). In addition, CVₗₙ was calculated using Equation (1) in the same manner as in Experimental Example 1.

TABLE 9-1

| | Plate 1 copy number of RNA | | | | |
|---|---|---|---|---|---|
| | 4 | 8 | 16 | 32 | 64 |
| Cq Ave | 37.36 | 36.20 | 35.28 | 34.36 | 33.37 |
| σ | 0.44 | 0.17 | 0.21 | 0.15 | 0.11 |
| CV % | 1.17 | 0.48 | 0.60 | 0.45 | 0.32 |
| Detection | 100.0% | 100.0% | 100.0% | 100.0% | 100.0% |
| rate | | | | | |
| CVₗₙ | 31.5% | 12.4% | 14.9% | 11.0% | 7.6% |

TABLE 9-2

| | Plate 2 copy number of RNA | | | | |
|---|---|---|---|---|---|
| | 4 | 8 | 16 | 32 | 64 |
| Cq Ave | 37.65 | 36.44 | 35.40 | 34.49 | 33.43 |
| σ | 0.47 | 0.27 | 0.24 | 0.16 | 0.11 |
| CV % | 1.24 | 0.73 | 0.68 | 0.46 | 0.32 |
| Detection | 100.0% | 100.0% | 100.0% | 100.0% | 100.0% |
| rate | | | | | |
| CVₗₙ | 31.8% | 17.8% | 16.1% | 10.6% | 7.1% |

As shown in FIG. 26, based on the comparison with the results of Experimental Example 1 (FIG. 24), the inhibitory effect of the components contained in the negative specimen was small, but the Cq value was likely to be deviated from the calibration curve due to the inhibitory effect of the components contained in the negative specimen as the copy number of BCR-ABL1 P210 decreased.

As shown in Tables 9-1 and 9-2, the limit of detection (LOD) was 4 copies of RNA, and the limit of quantitation (LOQ) was 4 copies of RNA.

The present invention includes the following aspects.

(1) A method of determining a limit of detection and a limit of quantitation in a nucleic acid detection test, the method including, by using a container which includes a negative control group consisting of one or more wells containing no target nucleic acids and a positive control group consisting of wells containing specific copy numbers of target nucleic acids and having three or more wells with different specific copy numbers of the target nucleic acids and in which at least one specific copy number of the target nucleic acids in the positive control group is 1 or greater and less than 100, adding a negative specimen that does not contain the target nucleic acids to the positive control group and adding a reagent used for a nucleic acid detection test to the negative control group and the positive control group to amplify the target nucleic acids, and determining a smallest specific copy number among specific copy numbers with a detection rate of 95% or greater in the positive control group as a limit of detection in the nucleic acid detection test and determining a smallest specific copy number among specific copy numbers with CVₗₙ of 35% or less, where CVₗₙ is represented by Equation (I), as a limit of quantitation in the nucleic acid detection test in a case where the target nucleic acids are not detected in the negative control group.

CVₗₙ=\sqrt{(1+E)^{(SD)Cq))}^{²}*^{ln(1+E)}−1} Equation (I):

In Equation (I), E represents an efficiency of a nucleic acid amplification reaction, SD (Cq) represents a standard deviation of Cq, and Cq represents the number of cycles at which an amplification curve and a threshold intersect in the nucleic acid amplification reaction.

(2) The determination method according to (1), in which at least three of the specific copy numbers of the target nucleic acids in the positive control group are 1 or greater and less than 100.

(3) The determination method according to (1) or (2), in which the positive control group has five or more wells with different specific copy numbers of the target nucleic acids.

(4) The determination method according to (3), in which at least five of the specific copy numbers of the target nucleic acids in the positive control group are 1 or greater and less than 100.

(5) The determination method according to any one of (1) to (4), in which the negative specimen is a nucleic acid extract from a healthy subject.

(6) The determination method according to any one of (1) to (5), in which the container consists of the wells containing the specific copy numbers of the target nucleic acids and further includes a well group for creating a calibration curve, which has three or more wells with different specific copy numbers of the target nucleic acids, and at least one specific copy number of the target nucleic acids in the well group for creating a calibration curve is 1 or greater and less than 100.

(7) The determination method according to any one of (1) to (6), in which the target nucleic acids are incorporated into nucleic acids in a nucleus of a cell.

(8) The determination method according to (7), in which the cell is a yeast.

(9) The determination method according to (7) or (8), in which the cell is jetted by an ink jet method.

While preferred embodiments of the invention have been described and illustrated above, it should be understood that these are exemplary of the invention and are not to be considered as limiting. Additions, omissions, substitutions, and other modifications can be made without departing from the scope of the invention. Accordingly, the invention is not to be considered as being limited by the foregoing description and is only limited by the scope of the appended claims.

### EXPLANATION OF REFERENCES

1: Container
2: Well containing no target nucleic acids
3: Negative control group
4, 4a, 4b, 4c, 4d: Well containing target nucleic acids
5: Positive Control group
10, 10′, 10C: Jet head (liquid droplet-jetting means)
11, 11a, 11b, 11c, 11C, 11′: Liquid chamber
12, 12C: Membrane
13, 13C: Driving element
13a: Electric motor
13b, 13c: Piezoelectric element
20: Driving means
30, 260: Light source
40: Mirror
60, 61: Light-receiving element
70: Control means
71, 101: CPU
72: ROM
73: RAM
74, 106: I/F
75: Bus line
111, 111a, 111b, 111c, 121: Nozzle
112: Electromagnetic valve
115: Atmosphere releasing unit
200: Coil
250: Microchannel
255: Detector
255′: Image acquisition unit
265, 265′: Lens
300, 300a, 300b, 300c: Cell suspension
310, 310′: Liquid droplet
350, 350a, 350b, 350′, 350″: Cell
400: Dispensing device
401, 401A, 401B, 401C: Liquid droplet-forming device
700, 700′: Plate
710: Well
800: Stage
900: Control device
L: Light
Lf, Lf₁, Lf₂: Fluorescence

### CITATION LIST

### Patent Documents

[Patent Document 1] Japanese Unexamined Patent Application, First Publication No. 2018-124171

[Patent Document 2] Japanese Unexamined Patent Application, First Publication No. 2017-187473

What is claimed is:

1. A method of determining a limit of detection and a limit of quantitation in a nucleic acid detection test, the method comprising:
obtaining a container which includes a negative control group consisting of one or more wells containing no target nucleic acids and a positive control group consisting of wells containing specific copy numbers of target nucleic acids and having three or more wells with different specific copy numbers of the target nucleic acids and in which at least one specific copy number of the target nucleic acids in the positive control group is 1 or greater and less than 100;
adding a negative specimen that does not contain the target nucleic acids to the positive control group and adding a reagent used for a nucleic acid detection test to the negative control group and the positive control group to amplify the target nucleic acids; and
determining a smallest specific copy number among specific copy numbers with a detection rate of 95% or greater in the positive control group as a limit of detection in the nucleic acid detection test and determining a smallest specific copy number among specific copy numbers with CVₗₙ of 35% or less, where CVₗₙ is represented by Equation (I), as a limit of quantitation in the nucleic acid detection test in a case where the target nucleic acids are not detected in the negative control group,
CVₗₙ=\sqrt{(1+E)^{(SD)Cq))}^{²}*^{ln(1+E)}−1} Equation (I):
wherein in Equation (I), E represents an efficiency of a nucleic acid amplification reaction, SD (Cq) represents a standard deviation of Cq, and Cq represents the number of cycles at which an amplification curve and a threshold intersect in the nucleic acid amplification reaction.

2. The determination method according to claim 1,
wherein at least three of the specific copy numbers of the target nucleic acids in the positive control group are 1 or greater and less than 100.

3. The determination method according to claim 1,
wherein the positive control group has five or more wells with different specific copy numbers of the target nucleic acids.

4. The determination method according to claim 3,
wherein at least five of the specific copy numbers of the target nucleic acids in the positive control group are 1 or greater and less than 100.

5. The determination method according to claim 1,
wherein the negative specimen is a nucleic acid extract from a healthy subject.

6. The determination method according to claim 1,
wherein the container comprises the wells containing the specific copy numbers of the target nucleic acids and a well group for creating a calibration curve, which has three or more wells with different specific copy numbers of the target nucleic acids, and
at least one specific copy number of the target nucleic acids in the well group for creating a calibration curve is 1 or greater and less than 100.

7. The determination method according to claim 1,
wherein the target nucleic acids are incorporated into nucleic acids in a nucleus of a cell.

8. The determination method according to claim 7,
wherein the cell is a yeast.

9. The determination method according to claim 7,
wherein the cell is jetted by an ink jet method.
